(19) United States
(12) Patent Application Publication
(10) Pub. No.: US 2020/0029897 A1
(43) Pub. Date: Jan. 30, 2020

(54) BIOMETRIC-INFORMATION MEASURING ELECTRODE AND METHOD FOR MEASURING BIOMETRIC INFORMATION

(71) Applicant: ALPS ALPINE CO., LTD., Tokyo (JP)

(72) Inventor: Isao TAKAHASHI, Miyagi-ken (JP)

(21) Appl. No.: 16/592,925

(22) Filed: Oct. 4, 2019

Related U.S. Application Data
(63) Continuation of application No. PCT/JP2018/011879, filed on Mar. 23, 2018.

(30) Foreign Application Priority Data
Apr. 7, 2017 (JP) 2017-076744

(51) Int. Cl. A61B 5/00

(52) U.S. Cl. CPC A61B 5/6801; A61B 2562/16; A61B 2562/04

(57) ABSTRACT

A biometric-information measuring electrode includes a conductive bundle including a plurality of conductive wires and having a distal end which is one end and a rear end which is another end, a terminal electrically connected to the conductive bundle, a support portion located adjacent to the distal end of the conductive bundle and movably supporting the conductive bundle, and a protrusion mechanism (a first protrusion mechanism) configured to protrude the conductive bundle toward the distal end. The distal end of the conductive bundle is capable of coming into contact with a living organism. The support portion keeps a relative position of the conductive bundle to the terminal in a state in which the protrusion mechanism is not performing an operation to protrude the conductive bundle. This allows the electrode to be repeatedly used even if the electrode is soiled.

### CLAIM OF PRIORITY

This application is a Continuation of International Application No. PCT/JP2018/011879 filed on Mar. 23, 2018, which claims benefit of Japanese Patent Application No. 2017-076744 filed on Apr. 7, 2017. The entire contents of each application noted above are hereby incorporated by reference.

### BACKGROUND OF THE INVENTION

### 1. Field of the Invention

The present invention relates to biometric-information measuring electrodes, such as an electroencephalographic electrode and an electrocardiographic measurement electrode, and a method for measuring biometric information using the biometric-information measuring electrodes.

### 2. Description of the Related Art

In recent years, measuring various biometric information, such as pulse waves, electrocardiograms, electromyograms, body fats, and electroencephalograms, has increased. At that time, various biometric-information measuring electrodes have been proposed for stable contact with the living organism. In particular, biometric-information measuring electrodes suitable for skins with hair, such as an electroencephalographic electrode, need to be in contact with the skin while avoiding the hair on the skin (a scalp in the case of electroencephalographic electrodes) as appropriate, in which case it is also required to reduce the burden on the skin. Furthermore, it is often required that the electrodes can be used multiple times.

Japanese Unexamined Patent Application Publication No. 2015-16166 discloses, as an electroencephalographic electrode that meets such issues, a biometric electrode (a biometric-information measuring electrode) characterized in that a plurality of contacts made of conductive fibers obtained by coating base fibers with a conductive polymer extend in a raised state from the base.

When the biometric electrode (the biometric-information measuring electrode) disclosed in Japanese Unexamined Patent Application Publication No. 2015-16166, in which the contacts are coated with conductive gel (or an electrolytic solution) in order to stabilize contact to the living organism, is to be reused, the conductive gel (or the electrolytic solution) adhering to the conductive fibers constituting the contacts has to be cleared.

Even if the conductive gel (or the electrolytic solution) is not used, contact with the living organism (skin) at the measurement may cause stains including the sweat, blood, lymph fluid, and other biological fluids, and the cuticles of the subject to adhere to the biometric electrode (the biometric-information measuring electrode). For this reason, the biometric electrode (the biometric-information measuring electrode) needs to be cleaned.

However, the work of cleaning for removing the conductive gel and adherents from the biometric electrode and sterilization has the problem of heavy work load. In particular, the biometric electrode (the biometric-information measuring electrode) disclosed in Japanese Unexamined Patent Application Publication No. 2015-16166 is difficult to clean and practically cannot be repeatedly used, so that it must be disposable.

### SUMMARY OF THE INVENTION

The present invention provides a biometric-information measuring electrode that can be repeatedly used even if the electrode is soiled. The present invention also provides a method for manufacturing the biometric-information measuring electrode.

In an aspect of the present invention, a biometric-information measuring electrode includes a columnar conductor conducting electricity between a distal end which is one end and a rear end which is another end, a terminal electrically connected to the columnar conductor, a support portion located adjacent to the distal end of the columnar conductor and movably supporting the columnar conductor, and a protrusion mechanism configured to protrude the columnar conductor toward the distal end. A portion (a contact portion) of the columnar conductor including the distal end is capable of coming into contact with a living organism. The support portion keeps a relative position of the columnar conductor to the terminal in a state in which the protrusion mechanism is not performing an operation to protrude the columnar conductor.

Since the biometric-information measuring electrode includes the support portion and the protrusion mechanism, the columnar conductor is movable in the direction in which the distal end of the columnar conductor protrudes, in which state, the columnar conductor can be fixed (held). This allows the columnar conductor to be pressed in the protruding direction after a conductive gel or the like is applied to the contact portion of the columnar conductor and to cut off the contact portion where remaining conductive gel or the like is present. Thus, a new contact portion of the columnar conductor is formed for the next measurement even without cleaning the contact portion. This makes it possible to use the biometric-information measuring electrode continuously, not throwing it away.

In the biometric-information measuring electrode, the protrusion mechanism may include a temporarily holding portion located nearer to the rear end than the support portion and capable of temporarily holding the columnar conductor and a displacement portion capable of changing a separation distance between the support portion and the temporarily holding portion by deforming the displacement portion itself. The temporarily holding portion may hold the columnar conductor stronger than the support portion when the columnar conductor moves toward the distal end in accordance with deformation of the displacement portion. When the protrusion mechanism performs an operation to protrude the columnar conductor, the displacement portion is deformed so as to decrease the separation distance between the support portion and the temporarily holding portion. Since the decrease in the distance between the support portion and the temporarily holding portion is performed, with the temporarily holding portion holding the columnar conductor stronger than the support portion, the columnar conductor slides with respect to the support portion to move toward the distal end. Thus, this protrusion mechanism allows the columnar conductor to be moved toward the distal end stably with a simple configuration.

In the biometric-information measuring electrode, the displacement portion may be movable in an extending direction of the columnar conductor. Since the displacement portion is movable, the external form of the biometric information measuring electrode can be maintained in a state in which the protrusion mechanism is not operated. Since a change in the external form of the biometric-information measuring electrode can change the condition of measurement of the living organism (skin), such a configuration contributes to improvement in measurement stability.

In the biometric-information measuring electrode, the displacement portion may include a portion composed of an elastic material. In the case where the displacement portion includes a portion composed of an elastic material, the displacement portion can be reversibly deformed by elastic deformation and elastic recovery.

In the biometric-information measuring electrode, the displacement portion may have a bellows structure including a stretch portion extending and contracting along the columnar conductor. In the case where the displacement portion has a portion with the bellows structure, the displacement portion can be reversibly deformed by the extension and contraction of the portion with the bellows structure.

In the case where the biometric-information measuring electrode has the bellows structure, the support portion is preferably capable of holding the columnar conductor stronger than the temporarily holding portion when the stretch portion is extended. First, the stretch portion is elastically extended to separate the support portion and the temporarily holding portion from each other. At that time, the columnar conductor is held stronger at the support portion than at the temporarily holding portion, for example, by gripping the support portion strong. This allows the columnar conductor to be slid with respect to the temporarily holding portion when the stretch portion is extended. Thereafter, the stretch portion is contracted by the elastic recovery of the stretch portion. At that time, by holding the columnar conductor stronger at the temporarily holding portion than at the support portion, the columnar conductor slides with respect to the support portion to move toward the distal end. This protrusion mechanism allows the columnar conductor to move toward the distal end stably with the particularly simple structure.

The biometric-information measuring electrode may further include an elastic support portion constituting at least part of the support portion and elastically supporting the columnar conductor. A portion of the columnar conductor adjacent to the distal end is supported by the elastic support portion. Therefore, when the distal end of the columnar conductor comes into contact with the living organism, the portion adjacent to the distal end (a portion including the contact portion) is elastically deformed by the elastic support portion to come into contact with the living organism. The elastic deformation allows the contact between the living organism and the distal end in contact with the living organism to be maintained in a suitable state.

In the biometric-information measuring electrode, the protrusion mechanism may include a pressing member located at a further rear portion than the rear end of the columnar conductor, and when moving the columnar conductor toward the distal end, the pressing member is preferably capable of coming into contact with the portion of the columnar conductor including the rear end. Since the pressing member can come into contact with the portion of the columnar conductor including the rear end when moving the columnar conductor toward the distal end, the pressing member pushes the columnar conductor. Thus, the columnar conductor moves toward the distal end. This protrusion mechanism allows the columnar conductor to be moved toward the distal end more stably with a simple configuration.

The biometric-information measuring electrode may further include at least one casing having an opening through which the portion of the columnar conductor including the distal end is passed and exposed. The support portion may be located inside the opening. The casing may cover a periphery of the portion of the columnar conductor including the distal end. The casing covering the periphery of the distal end of the conductive bundle prevents a portion of the columnar conductor other than the contact portion for use in measurement from coming into contact with the object to be measured including skin (living organism). Thus, this configuration allows repeated use of the biometric-information measuring electrode favorably from a hygiene viewpoint.

In the case where the biometric-information measuring electrode includes the casing, the casing may have a tapered shape with a smaller outer peripheral shape toward the distal end. The tapered shape of the casing makes it easy to bring the distal end into contact with the target living organism (skin). Thus, this configuration may increase the measurement stability.

The casing may have a shape extending in a long axis direction of the columnar conductor, and a protruding portion of the columnar conductor protruding from the casing toward the distal end and the casing may constitute an electrode pin. The structure of the electrode pin makes it easy to bring the electrode into contact with the target living organism (skin). Thus, this configuration may increase the measurement stability.

The casing may have electrical conductivity and may be electrically connected to the terminal. Since the casing has electrical conductivity and is electrically connected to the terminal, signals from the columnar conductor can be transmitted to the terminal with more stability. Thus, this configuration may increase the measurement stability.

The biometric-information measuring electrode may include a plurality of casings. In each casing, the columnar conductor supported by the support portion may be electrically connected to the terminal. With this configuration, the biometric-information measuring electrode includes a plurality of columnar conductors capable of measuring a living organism (skin). Thus, this configuration may increase the measurement stability.

The protrusion mechanism may be provided for each of the plurality of casings. This configuration allows the plurality of columnar conductors of the biometric-information measuring electrode to be individually protruded. Thus, this configuration may increase the measurement stability.

The protrusion mechanism may cooperatively operate at least two of the plurality of columnar conductors corresponding to the plurality of casings. This configuration allows at least two of the plurality of columnar conductors of the biometric-information measuring electrode to be protruded at the same time. Thus, this configuration may decrease a preparation time for measurement, which increases the measurement efficiency.

In the biometric-information measuring electrode, the columnar conductor may include a portion of a conductive bundle of a plurality of conductive wires. Since the conductive bundle includes a plurality of conductive wires, the contact pressure of the contact portion against the living organism can easily be increased. In this case, the conductive bundle may include a portion of the plurality of conductive wires bound with an adhesive material. Since the conductive bundle includes a portion of the plurality of conductive wires bound with an adhesive material, the conductive bundle is easy to maintain its cross-sectional shape and is hardly separated into the plurality of conductive wires in use. Thus, this configuration may increase the measurement stability.

In the biometric-information measuring electrode, the conductive wires may be made of carbon fibers. In this case, since the conductive wires are made of carbon fibers, a change in measurement conditions due to corrosion, etc. is unlikely to occur on the conductive wires as compared with a metal-based material. Thus, this configuration may increase the measurement stability.

In the biometric-information measuring electrode, at least part of an outer surface of the conductive bundle may be a coating material that binds the plurality of conductive wires. Since the conductive bundle includes the coating material, the conductive bundle is easy to maintain its cross-sectional shape and is hardly separated into the plurality of conductive wires in use. Thus, this configuration may increase the measurement stability.

In the case where the conductive bundle of the biometric-information measuring electrode includes the coating material, the coating material may adhere to the plurality of conductive wires to such an extent as to be stripped by sliding of the support portion and the conductive bundle performed in the operation of the protrusion mechanism protruding the conductive bundle. At least part of the coating material is stripped at the portion of the conductive bundle protruding from the support portion adjacent to the distal end (contact portion). Thus, this configuration may increase the measurement stability even if the coating material is an insulating material.

In the biometric-information measuring electrode, the columnar conductor may include a conductive resin member whose base material is synthetic resin, and a new distal end may be formed by cutting part of the conductive resin member. This may simplify the configuration of the columnar conductor. Furthermore, since the cut surface of the conductive resin member forms at least part of the distal end, the contact area can be relatively increased (for example, as compared with a case in which the columnar conductor has a portion of the conductive bundle). The conductive resin member may be an insulating synthetic resin in which a conductive substance is dispersed or a synthetic resin used as the base material has conductivity.

The conductive resin member may contain a conductive carbon material and a binder resin that binds the conductive carbon material. Since carbon materials, such as carbon particles, carbon fibers, and nano-carbon materials, have an affinity for the binder resin, the conductive resin member is easy to use.

The conductive resin member may have a conductive coating disposed so as to coat at least an end of the distal end. In this case, it is not an essential condition that the entire conductive resin member has conductivity, which facilitates forming the conductive resin member, for example, by general resin molding. In this case, the conductive coating may also be disposed so as to coat the entire conductive resin member.

The columnar conductor may have a notch on its outer peripheral surface. The notch may be configured, when a portion of the columnar conductor adjacent to the distal end is cut off, part of a surface of the notch constitutes part of a surface of a new distal end formed. The presence of the notch facilitates cutting the columnar conductor, improving the work efficiency. This may simplify another configuration of the biometric-information measuring electrode.

In the case where the columnar conductor includes the notch on the outer peripheral surface, the columnar conductor may include a columnar conductive resin member whose base material is synthetic resin, and the notch may be provided on an outer peripheral surface of the columnar conductive resin member. With this configuration, when the portion of the columnar conductive resin member adjacent to the distal end is cut, both of the surface of the notch and the cut surface of the columnar substrate have conductivity. This increases the conductivity of the newly formed distal end.

The columnar conductive resin member may contain a conductive carbon material and a binder resin that binds the conductive carbon material. Since carbon materials, such as carbon particles, carbon fibers, and nano-carbon materials, have an affinity for the binder resin, the columnar conductive resin member is easy to use.

In the case where the columnar conductor has a notch on the outer peripheral surface, the columnar conductor may have a coated structure including a columnar substrate and a conductive coating that coats the columnar substrate, and the notch may be disposed on the outer peripheral surface of the coated structure. When the portion of the columnar conductor adjacent to the distal end is cut at the notch, the conductive coating is located also on part of the surface of the notch. This increases the conductivity of the surface of the newly formed distal end, thereby decreasing the contact impedance to the living organism.

The columnar conductor may include a combined member in which a plurality of individual contact members are detachably combined in a long axis direction of the columnar conductor. In this case, the combined member may constitute a portion of the columnar conductor adjacent to the distal end, and the individual contact member at the distal end of the combined member may be conducting to the terminal. This allows the end of the individual contact member adjacent to the distal end at the distal end of the combined member functions suitably as the distal end of the columnar conductor. With the combined member in which the columnar conductor is made of a plurality of individual contact members, by separating the individual contact member at the distal end of the columnar conductor, a columnar conductor having a new distal end can easily be provided.

In the case where the columnar conductor includes the combined member in which the columnar conductor is made of a plurality of individual contact members, the individual contact members of the combined member may have a fitted structure. The individual contact members adjacent in the long axis direction of the columnar conductor may be detachably combined using the fitted structure. This configuration allows providing a columnar conductor having a new distal end particularly easily by detaching the individual contact member of the columnar conductor at the distal end of the combined member from an individual contact member combined to the individual contact member.

In the case where the columnar conductor includes the combined member in which the columnar conductor is made of a plurality of individual contact members, the combined member may include a core material extending in the long axis direction of the columnar conductor. In this case, when the core material is disposed so as to pass through a plurality of the individual contact members, a columnar conductor having a new distal end can be provided particularly easily by detaching an individual contact member at the distal end of the columnar conductor. In this case, in cutting the individual contact member, it is preferable to cut part of the core material located at the distal end of the columnar conductor from the viewpoint of keeping the distal end of the individual contact members clean.

In the case of using the core material, the core material preferably has electrical conductivity. This allows collecting biometric information not only on the surface of the individual contact member adjacent to the distal end of the columnar conductor but also on the surface (the cross section) of the core material adjacent to the distal end of the columnar conductor.

An individual substrate that is a base of each individual contact member may be a conductive resin member whose base material is synthetic resin. This may ensure the conduction between the combined individual contact members more stably.

The conductive resin member may contain a conductive carbon material and a binder resin that binds the conductive carbon material. Since carbon materials, such as carbon particles, carbon fibers, and nano-carbon materials, have an affinity for the binder resin, the conductive resin member is easy to use.

The individual contact member may have a conductive coating disposed so as to coat at least an end of the conductive resin member adjacent to the distal end. In this case, the conductive coating may be disposed so as to coat the entire conductive resin member. This configuration may ensure the conduction between the combined individual contact member more stably.

An individual substrate that is a base of each individual contact member may be an insulating material. A conductive coating may be disposed so as to coat the entire insulating material. In this case, the individual substrate can be formed using general resin molding, which makes it easy to form the individual substrate. A material, such as an acrylic resin, polyolefin, or polyester, which is insulative but can be used in a manufacturing method capable of shaping, such as injection molding, easily and highly accurately, can be used as a constituent material of the individual substrate.

The conductive coating preferably includes conductive polymer, such as poly-3,4-ethylenedioxythiophene (PEDOT) from the viewpoint of increasing the stability between the distal end of the columnar conductor and the living organism.

The columnar conductor may be elastically deformable. In the case where the columnar conductor is elastically deformable, by forming a conductive coating on the surface of the columnar conductor in a state in which a tensile force is applied in the direction of the long axis of the columnar conductor to extend the columnar conductor, the conductive coating can easily be formed at the end of the columnar conductor adjacent to the distal end when the tensile force is cancelled to recover the shape before extension.

In another aspect of the present invention, a method for measuring biometric information using the biometric-information measuring electrode is provided. The method includes the steps of protruding part of the columnar conductor toward the distal end to form an additional protruding portion by operating the protrusion mechanism, removing part of the columnar conductor in such a manner that at least part of the additional protruding portion protruded in the protruding step constitutes a cut-remaining component of the biometric-information measuring electrode, and after the removing step, measuring biometric information by bringing the portion of the columnar conductor protruding toward the distal end into contact with the living organism. Since the columnar conductor is protruded in the protruding step and is cut appropriately in the removing step, measurement of the living organism (skin) can be performed using a portion of the columnar conductor not used in the measuring step in the measuring step. Thus, this configuration may increase the measurement stability and allow hygienic measurement.

### BRIEF DESCRIPTION OF THE DRAWINGS

FIG. 1 is a cross-sectional view of a biometric-information measuring electrode according to a first embodiment of the present invention conceptually illustrating the structure;

FIG. 2 is a cross-sectional view of the biometric-information measuring electrode according to the first embodiment of the present invention conceptually illustrating a protruding step of operating a protrusion mechanism using a first method;

FIG. 3 is a cross-sectional view of the biometric-information measuring electrode according to the first embodiment of the present invention conceptually illustrating a state in which a conductive bundle is protruded by operating the protrusion mechanism;

FIG. 4 is a cross-sectional view of the biometric-information measuring electrode according to the first embodiment of the present invention conceptually illustrating a state in which refresh processing on the conductive bundle including operating the protrusion mechanism has been completed;

FIG. 5 is a cross-sectional view of the biometric-information measuring electrode according to the first embodiment of the present invention conceptually illustrating a state in which a protrusion mechanism is operated using a second method;

FIG. 6 is a cross-sectional view of the biometric-information measuring electrode according to the first embodiment of the present invention conceptually illustrating a state in which a protrusion mechanism is operated using a third method;

FIG. 7 is a cross-sectional view of the biometric-information measuring electrode according to the first embodiment of the present invention conceptually illustrating a state in which the conductive bundle is protruded by operating the protrusion mechanism using the third method;

FIG. 8 is a cross-sectional view of a biometric-information measuring electrode according to a second embodiment of the present invention conceptually illustrating the structure;

FIG. 9 is a cross-sectional view of a biometric-information measuring electrode according to a third embodiment of the present invention conceptually illustrating the structure;

FIG. 10A is an external view of a biometric-information measuring electrode (an electrode unit) according to a fourth embodiment of the present invention conceptually illustrating the structure;

FIG. 10B is a cross-sectional view taken along line XB-XB of FIG. 10A;

FIG. 11A is an external view of a biometric-information measuring electrode (an electrode unit) according to a fifth embodiment of the present invention conceptually illustrating the structure;

FIG. 11B is a cross-sectional view taken along line XIB-XIB of FIG. 11A;

FIG. 12 is a cross-sectional view of a biometric-information measuring electrode according to a sixth embodiment of the present invention conceptually illustrating the structure;

FIG. 13 is a cross-sectional view of a biometric-information measuring electrode according to a seventh embodiment of the present invention conceptually illustrating the structure;

FIG. 14 is a cross-sectional view of the biometric-information measuring electrode according to the seventh embodiment of the present invention conceptually illustrating a state in which a protrusion mechanism is being operated;

FIG. 15 is a cross-sectional view of the biometric-information measuring electrode according to the seventh embodiment of the present invention illustrating a state in which the conductive bundle is protruded by operating the protrusion mechanism;

FIG. 16A is a cross-sectional view of a first modification of the coated conductive bundle that may be included in a biometric-information measuring electrode according to an embodiment of the present invention, conceptually illustrating the structure;

FIG. 16B is a cross-sectional view of a second modification of the coated conductor bundle, conceptually illustrating the structure;

FIG. 17A is an external view of a third modification of the coated conductor bundle, conceptually illustrating the structure;

FIG. 17B is a conceptual cross-sectional view of the third modification of the coated conductor bundle;

FIG. 18A is an external view of a fourth modification of the coated conductor bundle, conceptually illustrating the structure;

FIG. 18B is a conceptual cross-sectional view of the fourth modification of the coated conductor bundle;

FIG. 19 is a cross-sectional view of a biometric-information measuring electrode according to an eighth embodiment of the present invention conceptually illustrating the structure thereof;

FIG. 20 is a front view of a fifth modification of a conductive resin member that may be included in a biometric-information measuring electrode according to an embodiment of the present invention;

FIG. 21 is a cross-sectional view of a sixth modification of the conductive resin member;

FIG. 22 is a front view of a seventh modification of the conductive resin member;

FIG. 23 is a cross-sectional view of a portion enclosed by the one-dot chain line taken along line XXIII in FIG. 22;

FIG. 24 is a front view of an eighth modification of the conductive resin member; and

FIG. 25 is a cross-sectional view of a portion enclosed by the one-dot chain line XXV in FIG. 24.

### DESCRIPTION OF THE PREFERRED EMBODIMENTS

Embodiments of the present invention will be described hereinbelow with reference to the drawings. In the following description, like components are denoted by like reference signs, and descriptions of components once described are omitted as appropriate.

### First Embodiment

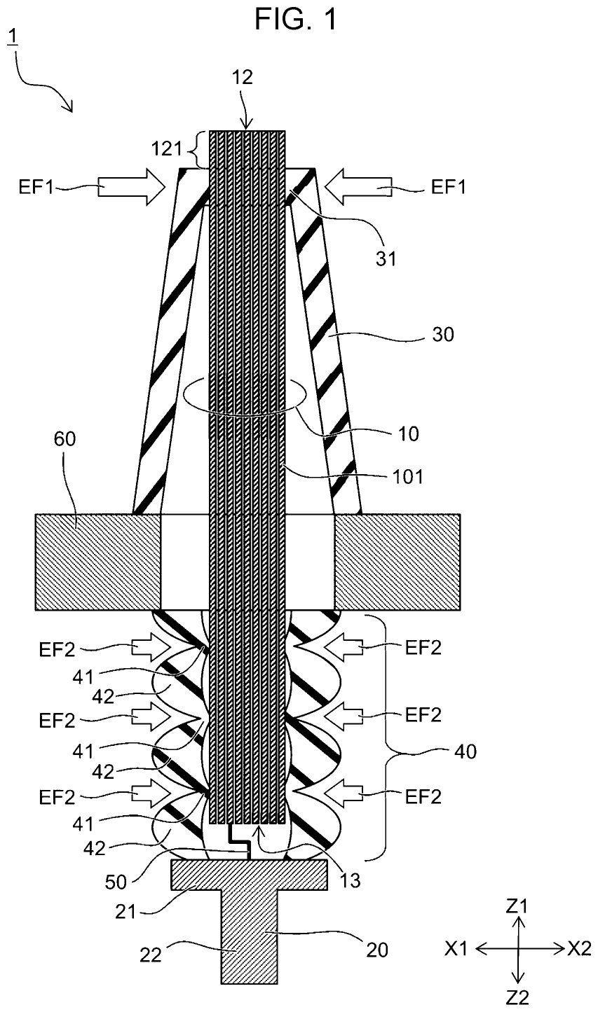

FIG. 1 is a cross-sectional view of a biometric-information measuring electrode 1 according to a first embodiment of the present invention conceptually illustrating the structure.

As illustrated in FIG. 1, the biometric-information measuring electrode 1 according to the first embodiment of the present invention includes a columnar conductor that conducts electricity between a distal end 12, which is one end (an end on the Z1 side in the Z1-Z2 direction) and a rear end 13, which is the other end (an end on the Z2 side in the Z1-Z2 direction), and a terminal 20 electrically connected to the columnar conductor. Between the columnar conductor and the terminal 20A, a wiring line 50 that is electrically connected thereto is disposed. In the first embodiment of the present invention, the columnar conductor is an extending conductive bundle 10 including a plurality of conductive wires 101.

This may make it easy to increase the contact pressure of a contact portion 121 at the distal end 12 of the conductive bundle 10 in measuring biometric information by pressing the distal end 12 against the living organism, improving the stability of measurement. Moreover, if each of the conductive wires 101 of the conductive bundle 10 is 0.3 mm or less, the effect of passing through the horny layer on the surface of the skin and stains adhering to the skin including exfoliated cuticles is expected. This may further improve the stability of measurement due to reduction in living organism contact impedance. In FIG. 1, the direction in which the conductive bundle 10 extends (extending direction) is expressed as “Z1-Z2 direction”.

The conductive wires 101 of the conductive bundle 10 of the biometric-information measuring electrode 1 may be fibers (carbon fibers) composed of a conductive carbon material. In the biometric-information measuring electrode 1, a portion of the conductive bundle 10 adjacent to the distal end 12 is a contact portion 121 to come into contact with the living organism. The contact portion 121 comes into contact with the living organism to collect biometric information as electrical signals from the living organism. The constituent material of the conductive wires 101 may be any conductive material. However, the carbon fibers are suitable for the constituent material of the biometric-information measuring electrode 1 because of its electrical conductivity and affinity for living organisms. Furthermore, the carbon fibers less change in measurement condition due to corrosion or the like than metal conductive wires 101. Thus, since the conductive wires 101 are made of carbon fibers, the measurement stability of the biometric-information measuring electrode 1 may be improved.

The biometric-information measuring electrode 1 includes a support portion 31 located at the distal end 12 of the conductive bundle 10 (on the Z1 side in the Z1-Z2 direction) and movably supporting the conductive bundle 10. In the biometric-information measuring electrode 1, the support portion 31 is an elastic support portion entirely composed of an elastic material and elastically supporting the conductive bundle 10. An elastic supporting force of the support portion 31, denoted by sign EF1 in FIG. 1, is referred to as a first supporting force (hereinafter referred to as “first supporting force EF1”).

The support portion 31 is located on the Z1 side of a first elastic member 30 shaped like a substantially truncated cone in overall shape whose outside diameter decreases toward the Z1 side in the Z1-Z2 direction. The first elastic member 30 has a hollow passing therethrough in the Z1-Z2 direction, in which part of the conductive bundle 10 is located. The contact portion 121 of the conductive bundle 10 is a portion of the conductive bundle 10 passing through the through-hole of the first elastic member 30 adjacent to the Z1 side in the Z1-Z2 direction and protruding outward.

Thus, the support portion 31 is a portion of the first elastic member 30 located around the rim of the through-hole on the Z1 side in the Z1-Z2 direction. Since the support portion 31 is composed of an elastic material, the strength of the first supporting force EF1 that supports the support portion 31 in refresh processing (described below) can easily be changed. When the contact portion 121 of the conductive bundle 10 comes into contact with the living organism, a portion of the conductive bundle 10 adjacent to the distal end 12 (a portion including the contact portion 121) is elastically deformed by the elastic support portion 31 to come into contact with the living organism. The elastic deformation allows the contact between the living organism and the contact portion 121 in contact with the living organism to be maintained in a suitable state.

A ring-shaped rigid base member 60 having a through-hole in the Z1-Z2 direction is provided at an end of the first elastic member 30 on the Z2 side in the Z1-Z2 direction so as not to be separated from the first elastic member 30. Part of the conductive bundle 10 that continues from the first elastic member 30 is located in the through-hole of the base member 60.

A tubular second elastic member 40 having a hollow passing to the Z1 side and the Z2 side in the Z1-Z2 direction and extending in the Z1-Z2 direction is provided at an end of the base member 60 on the Z2 side in the Z1-Z2 direction so as not to be separated from the base member 60. Part of the conductive bundle 10 continuing from the base member 60 is located in the hollow of the second elastic member 40. An end of the conductive bundle 10 on the Z2 side in the Z1-Z2 direction is located in the hollow of the second elastic member 40. In other words, an end of the second elastic member 40 on the Z2 side in the Z1-Z2 direction and an end of the conductive bundle 10 on the Z2 side in the Z1-Z2 direction are separate from each other in the Z1-Z2 direction.

The second elastic member 40 may include temporarily holding portions 41 located on the Z2 side in the Z1-Z2 direction from the support portion 31 and capable of temporarily holding the conductive bundle 10 and elastic displacement portions 42 capable of changing the distance between the support portion 31 and the temporarily holding portions 41 by deforming the displacement portions 42 themselves. In FIG. 1, the temporarily holding portions 41 are in contact with the conductive bundle 10 similarly to the support portion 31. However, their supporting forces (indicated by arrows EF2 in FIG. 1, hereinafter referred to as “second supporting forces”) are lower than the first supporting force EF1 of the support portion 31.

A terminal 20 protruding to the Z2 side in the Z1-Z2 direction is provided at an end of the second elastic member 40 on the Z2 side in the Z1-Z2 direction. The terminal 20 is composed of an electrically conductive material and includes a main body 22 located on the Z2 side in the Z1-Z2 direction and a flange 21 connected from the main body 22 on the Z1 side in the Z1-Z2 direction and protruding in an X-Y plane. The terminal 20 is in contact with the second elastic member 40 at the flange 21 so as not to be separated. The material of the terminal 20 may be any conductive material. From the viewpoint of affinity for living organisms, the terminal 20 is preferably composed of a conductive carbon material or a metal-based material, such as copper, plated with gold.

In the biometric-information measuring electrode 1 with the above structure, the position of the conductive bundle 10 relative to the terminal 20 in a normal operation mode (a mode in which a protrusion mechanism described below is not operated, hereinafter referred to as “normal mode”) is kept by the first supporting force EF1 of the support portion 31. Also, the temporarily holding portions 41 are in contact with the conductive bundle 10 by the elastic returning force, but their supporting forces (the second supporting forces EF2) are lower than the first supporting force EF1.

A method for measuring biometric information using the biometric-information measuring electrode 1 will be described hereinbelow.

From the viewpoint of collecting biometric information from the living organism with more stability, the biometric-information measuring electrode 1 sometimes measures biometric information in a state in which a conductive gel or the like is applied to the living organism so that the conductive gel or the like is present between the contact portion 121 of the conductive bundle 10 of the biometric-information measuring electrode 1 and the living organism, as described above. Such measurement causes the conductive gel or the like to adhere to the plurality of conductive wires 101 constituting the conductive bundle 10 located at the contact portion 121. The use of the biometric-information measuring electrode 1, for new measurement, in which part of the conductive gel or the like applied to the living organism adhered to the plurality of conductive wires 101 at the contact portion 121 is not preferable from a hygiene viewpoint. For this reason, to reuse the biometric-information measuring electrode 1, the conductive gel or the like adhering to the plurality of conductive wires 101 at the contact portion 121 needs to be removed. In conventional biometric electrodes, it was common to remove the conductive gel or the like attached by measurement, but this work requires much time and effort and the cleaning liquid becomes waste, placing a burden on the operator and increasing the cost.

In contrast, the method for measuring biometric information using the biometric-information measuring electrode 1 includes refresh processing including a protruding step and a removing step, as will be described next, prior to a measuring step of measuring biometric information by bringing a portion (the contact portion 121) of the conductive bundle 10 protruding toward the distal end 12 into contact with the living organism. The refresh processing allows the plurality of conductive wires 101 constituting the contact portion 121 to which conductive gel or the like adheres to be removed using a simple method to prepare a new contact portion 121.

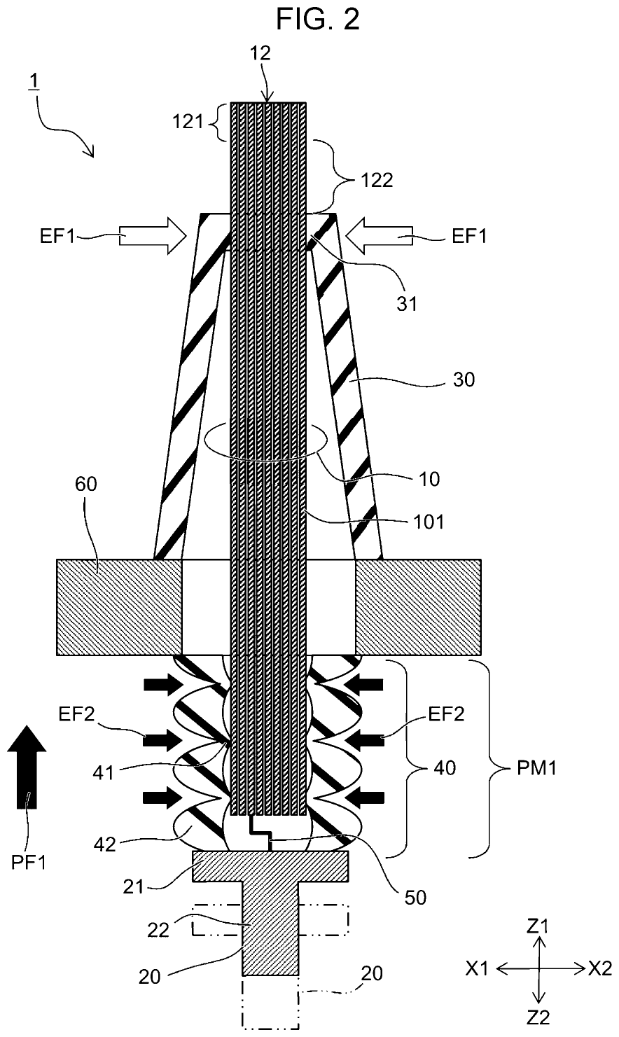

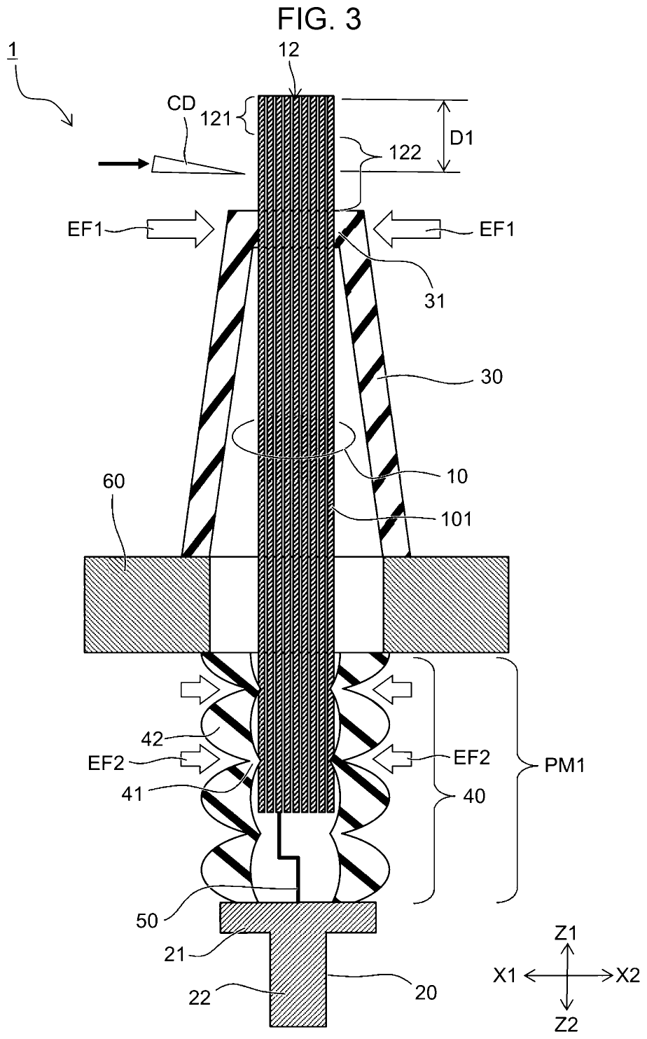

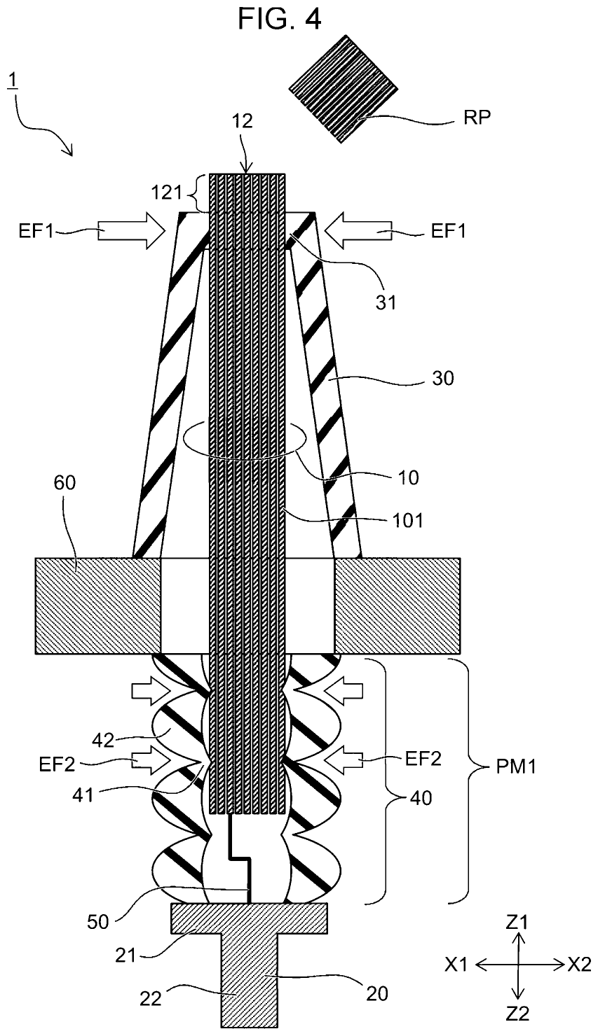

An example of the refresh processing will be described hereinbelow with reference to FIGS. 2 to 4. FIG. 2 is a cross-sectional view of the biometric-information measuring electrode 1 according to the first embodiment of the present invention conceptually illustrating the protruding step of operating a protrusion mechanism using a first method. FIG. 3 is a cross-sectional view of the biometric-information measuring electrode 1 according to the first embodiment of the present invention conceptually illustrating a state in which the conductive bundle 10 is protruded by operating the protrusion mechanism. FIG. 4 is a cross-sectional view of the biometric-information measuring electrode 1 according to the first embodiment of the present invention conceptually illustrating a state in which the refresh processing on the conductive bundle 10 including operating the protrusion mechanism has been completed.

The refresh processing includes the protruding step and the removing step described below.

As illustrated in FIG. 2, in the protruding step, the entire conductive bundle 10 of the biometric-information measuring electrode 1 is moved to the Z1 side in the Z1-Z2 direction to provide an additional protruding portion 122 additionally protruding from the support portion 31 to the Z1 side in the Z1-Z2 direction. Specifically, a pressing force is applied from the outer periphery of the second elastic member 40 to make the second supporting forces EF2 to be applied to the conductive bundle 10 at the temporarily holding portions 41 stronger than the first supporting force EF1 applied to the conductive bundle 10 at the support portion 31. FIG. 2 illustrates the second supporting forces EF2 in filled arrows, indicating that the second supporting forces EF2 are increased.

In this state, an external force to compress the second elastic member 40 to the Z1 side in the Z1-Z2 direction is applied. In FIG. 2, this external force is expressed as a first external force PF1. Since the entire second elastic member 40 constitutes the displacement portions 42 composed of an elastic material, the entire second elastic member 40 (the displacement portions 42) is deformed by the first external force PF1 to decrease the distance between the support portion 31 and the temporarily holding portions 41.

Since the second supporting forces EF2 are stronger than the first supporting force EF1, the frictional resistance of the conductive bundle 10 at the temporarily holding portions 41 is higher than the frictional resistance of the conductive bundle 10 at the support portion 31. Therefore, when the separation distance between the support portion 31 and the temporarily holding portions 41 is decreased, the first external force PF1 is transmitted from the temporarily holding portions 41 to the conductive bundle 10. As a result, the conductive bundle 10 slides with respect to the support portion 31 to protrude to the Z1 side in the Z1-Z2 direction. FIG. 2 illustrates a state in which part of the conductive bundle 10 is located as the additional protruding portion 122 nearer to the Z1 side in the Z1-Z2 direction from the support portion 31. The terminal 20 is also moved to the Z1 side in the Z1-Z2 direction by application of the first external force PF1. FIG. 2 shows the position of the terminal 20 before being moved using a two-dot chain line.

After the conductive bundle 10 is moved to the Z1 side in the Z1-Z2 direction in this manner, the application of the external forces (the first external force PF1 that compresses the second elastic member 40) to the conductive bundle 10 at the temporarily holding portions 41 is terminated. Then, as illustrated in FIG. 3, the strength of the second supporting forces EF2 returns to the strength in the normal mode, so that the frictional resistance of the conductive bundle 10 at the support portion 31 becomes higher than the frictional resistance of the conductive bundle 10 at the temporarily holding portions 41. At that time, the conductive bundle 10 slides with respect to the temporarily holding portions 41 while being supported at the support portion 31, so that the degree of protrusion of the distal end 12 of the conductive bundle 10 to the Z1 side in the Z1-Z2 direction is maintained. Since the first external force PF1 is not applied, the second elastic member 40 is elastically recovered to the Z2 side in the Z1-Z2 direction, so that the external shape of the second elastic member 40 returns to the shape in the normal mode, and the terminal 20 also returns to the position in the normal mode.

In the above protruding step, the second elastic member 40 makes the conductive bundle 10 protrude to the Z1 side in the Z1-Z2 direction in cooperation with the support portion 31. Thus, in the first method performed in the protruding step, the second elastic member 40 serves as a protrusion mechanism (a first protrusion mechanism PM1).

Subsequently, the removing step of cutting and removing part of the conductive bundle 10 is performed so that at least part of the additional protruding portion 122 protruded in the protruding step constitutes a cut-remaining component of the biometric-information measuring electrode 1. Specifically, as illustrated in FIG. 3, a portion with a length of Dl of the conductive bundle 10 from an end on the Z1 side in the Z1-Z2 direction to the Z2 side is cut with a cutting device CD. As a result, the biometric-information measuring electrode 1 in which a new contact portion 121 protrudes from the support portion 31 is obtained as illustrated in FIG. 4.

If the portion cut with the cutting device CD includes part of the additional protruding portion 122, the contact portion 121 of the conductive bundle 10 newly set at the cut remaining portion does not include the portion used in preceding measurement. This eliminates a possibility that a conductive gel or the like adheres to the new contact portion 121, and it is preferable. The above removing step removes the plurality of conductive wires 101 located at the end of the conductive bundle 10 on the Z1 side in the Z1-Z2 direction, to which a conductive gel or the like adheres, is removed from the biometric-information measuring electrode 1. FIG. 4 illustrates the removed portion RP of the conductive bundle 10. The method of removal may be mechanical cutting or cutting using a laser beam.

As a result of the refresh processing, as illustrated in FIG. 4, the biometric-information measuring electrode 1 is obtained in which a predetermined length of the contact portion 121 of the conductive bundle 10 protrudes from the first elastic member 30, which is similar in appearance to that in the normal mode illustrated in FIG. 1. Although the difference from the biometric-information measuring electrode 1 illustrated in FIG. 1 is only the length of the conductive bundle 10 in the Z1-Z2 direction (in the extending direction), there is no difference in appearance from the biometric-information measuring electrode 1 illustrated in FIG. 1. Thus, since the displacement portions 42 are composed of an elastic material, the displacement portions 42 can be reversibly deformed by elastic deformation and elastic recovery. In other words, the displacement portions 42 are movable (extensible) in the extending direction of the conductive bundle 10 (in the Z1-Z2 direction) and can return to the state before deformation. Thus, the biometric-information measuring electrode 1 changed in external form only while the protrusion mechanism (the first protrusion mechanism PM1) is operated, but in the normal mode, the external form of the biometric-information measuring electrode 1 can be maintained. Since a change in the external form of the biometric-information measuring electrode 1 can change the condition of measurement of the living organism (skin), such a configuration contributes to improvement in measurement stability.

The plurality of conductive wires 101 constituting the contact portion 121 of the conductive bundle 10 after the refresh processing have no conductive gel or the like attached because the conductive wires 101 are located inside the first elastic member 30 before the refresh processing. Therefore, the biometric-information measuring electrode 1 illustrated in FIG. 4 can perform the measuring step for measuring the biometric information by bringing the portion (the new contact portion 121) of the conductive bundle 10 protruding to the distal end 12 into contact with the living organism immediately after the removing step in the refresh processing. In other words, the refresh processing allows the biometric-information measuring electrode 1 to be repeatedly used.

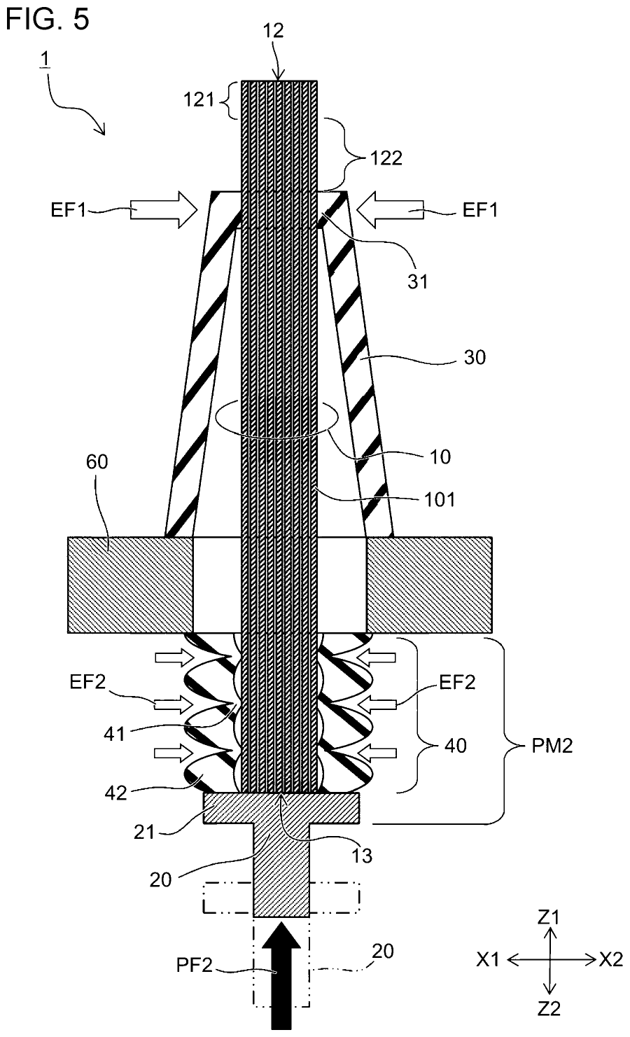

The refresh processing can be performed also by using a different method using the biometric-information measuring electrode 1. FIG. 5 is a cross-sectional view of the biometric-information measuring electrode 1 according to the first embodiment of the present invention conceptually illustrating a state in which a protrusion mechanism is operated using a second method.

In the first method illustrated in FIG. 2, the first protrusion mechanism PM1 is provided by the second elastic member 40, and the first external force PF1 applied to the second elastic member 40 is transmitted to the conductive bundle 10 through the temporarily holding portions 41. In the second method illustrated in FIG. 5, the flange 21 of the terminal 20 is brought into contact with the rear end 13 of the conductive bundle 10 (the end on the Z2 side in the Z1-Z2 direction) to directly apply an external force (a second external force PF2) toward the Z1 side in the Z1-Z2 direction to the conductive bundle 10. In FIG. 5, the terminal 20 before being moved is indicated by the two-dot chain line so as to make it easy to understand that the terminal 20 is moved to the Z1 side in the Z1-Z2 direction by the second external force PF2.

The application of the second external force PF2 causes the conductive bundle 10 to slide with respect to the support portion 31 and the temporarily holding portions 41 to protrude to the Z1 side in the Z1-Z2 direction. Thus, in the second method, the flange 21 of the terminal 20 serves as a pressing member to directly push the conductive bundle 10 without using the temporarily holding portions 41. Accordingly, the protrusion mechanism (a second protrusion mechanism PM2) in this method includes the flange 21 of the terminal 20. This second protrusion mechanism PM2 allows the conductive bundle 10 to be moved toward the distal end 12 more stably with a simple configuration.

Upon completion of the protrusion of the conductive bundle 10 by operating the second protrusion mechanism PM2, the application of the second external force PF2 is terminated. Then, the conductive bundle 10 supported at the support portion 31 because the first supporting force EF1 at the support portion 31 is stronger than the second supporting forces EF2 at the temporarily holding portions 41, and the conductive bundle 10 slides with respect to the temporarily holding portions 41 into the state illustrated in FIG. 3. Subsequent operations are common to those in the refresh processing including the first method, in which the portion with a length of Dl of the conductive bundle 10 from the distal end 12 to the Z2 side in the Z1-Z2 direction is removed to provide the biometric-information measuring electrode 1 having the new contact portion 121 of the conductive bundle 10, as illustrated in FIG. 4.

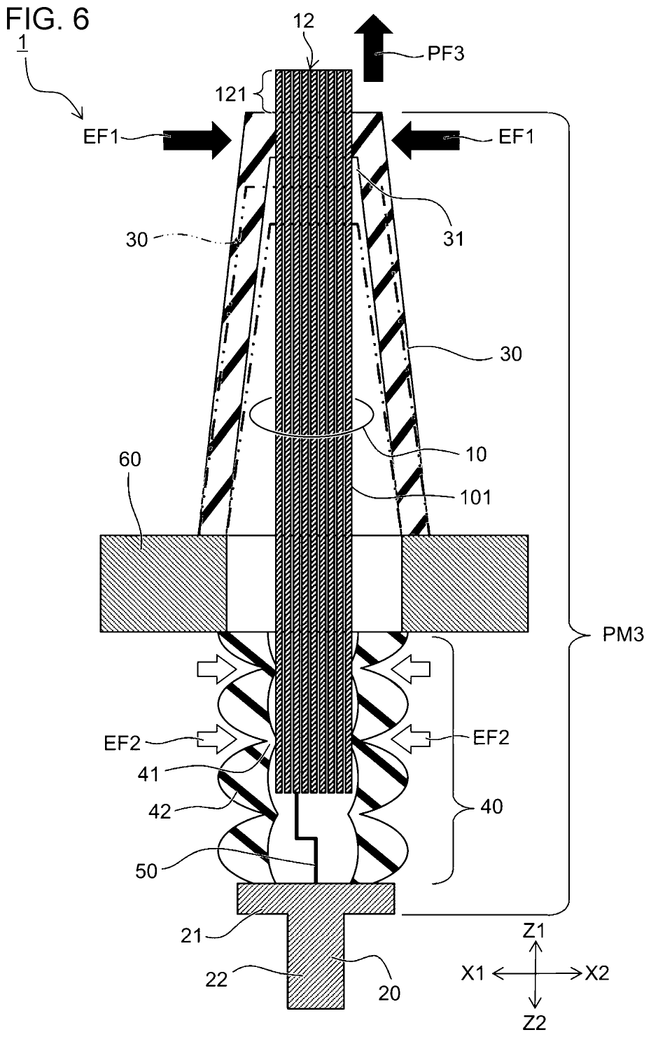

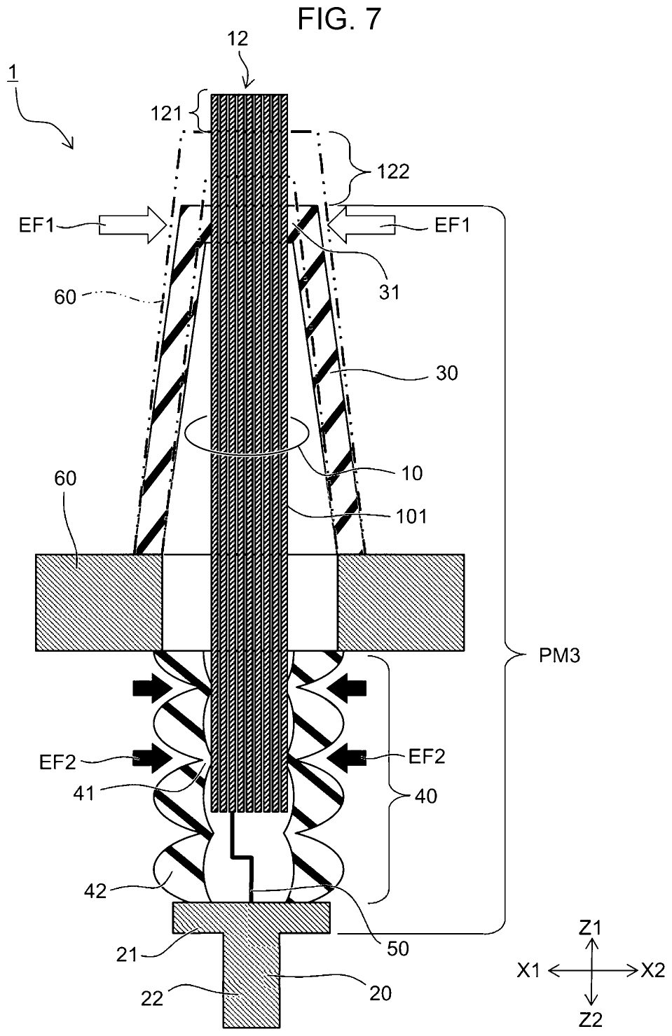

The refresh processing can be performed also by using another different method using the biometric-information measuring electrode 1. FIG. 6 is a cross-sectional view of the biometric-information measuring electrode 1 according to the first embodiment of the present invention conceptually illustrating a state in which a protrusion mechanism is operated using a third method. FIG. 7 is a cross-sectional view of the biometric-information measuring electrode 1 according to the first embodiment of the present invention conceptually illustrating a state in which the conductive bundle 10 is protruded by operating the protrusion mechanism using the third method.

The third method includes elastically deforming the first elastic member 30 to protrude the conductive bundle 10. As illustrated in FIG. 6, first, the strength of the first supporting force EF1 applied to the conductive bundle 10 at the support portion 31 including the end of the first elastic member 30 on the Z1 side in the Z1-Z2 direction is increased to increase the frictional resistance of the conductive bundle 10 at the support portion 31, and in this state, an external force (a third external force PF3) to move the support portion 31 to the Z1 side in the Z1-Z2 direction is applied. FIG. 6 illustrates the increase in the first supporting force EF1 using solid arrows.

By applying the third external force PF3, the first elastic member 30 is elastically deformed to extend to the Z1 side in the Z1-Z2 direction. FIG. 6 illustrates the first elastic member 30 in a state before the third external force PF3 is applied (in the normal mode) using two-dot chain lines for ease of understanding the elastic deformation. As the first elastic member 30 is elastically deformed, the entire conductive bundle 10 is also moved to the Z1 side in the Z1-Z2 direction. At the movement, the conductive bundle 10 slides at the temporarily holding portions 41 of the second elastic member 40.

Next, the strength of the second supporting forces EF2 to be applied from the temporarily holding portions 41 of the second elastic member 40 to the conductive bundle 10 is increased. FIG. 7 illustrates the increase in the second supporting force EF2 using solid arrows. Then, the strength of the first supporting force EF1 applied to the conductive bundle 10 at the support portion 31 is returned to the normal mode, and the application of the third external force PF3 is also cancelled. FIG. 7 illustrates the returning of the first supporting force EF1 to the normal mode using white arrows.

As a result, the first elastic member 30 elastically restores the shape in the normal mode. FIG. 7 illustrates the first elastic member 30 in a state before the third external force PF3 is applied (in the normal mode) using two-dot chain lines for ease of understanding the elastic deformation. As the first elastic member 30 is elastically restored, the support portion 31 moves to the Z2 side in the Z1-Z2 direction. Although the strength of the second supporting forces EF2 has been increased, as described above, the first supporting force EF1 is in the normal mod2, so that the force of friction against the conductive bundle 10 is stronger at the temporarily holding portions 41 than at the support portion 31. For this reason, the conductive bundle 10 slides with respect to the support portion 31, and as a result, the conductive bundle 10 protrudes to the Z1 side in the Z1-Z2 direction with respect to the support portion 31 returned to the position in the normal mode, as illustrated in FIG. 7. When the second supporting forces EF2 is returned to the strength in the normal mode, the conductive bundle 10 goes to the state illustrated in FIG. 3. Since subsequent operations are common to those of the first method including the refresh processing, a description thereof will be omitted.

Thus, the third method protrudes the portion of the conductive bundle 10 including the distal end 12 to the Z1 side in the Z1-Z2 direction using the elastic deformation and elastic recovery of the first elastic member 30. Accordingly, in the third method included in this refresh processing, the portion from the first elastic member 30 to the second elastic member 40 serves as a protrusion mechanism (a third protrusion mechanism PM3). This third protrusion mechanism PM3 allows the conductive bundle 10 to be stably moved toward the distal end 12 (to the Z1 side in the Z1-Z2 direction) with a simple configuration.

### Second Embodiment

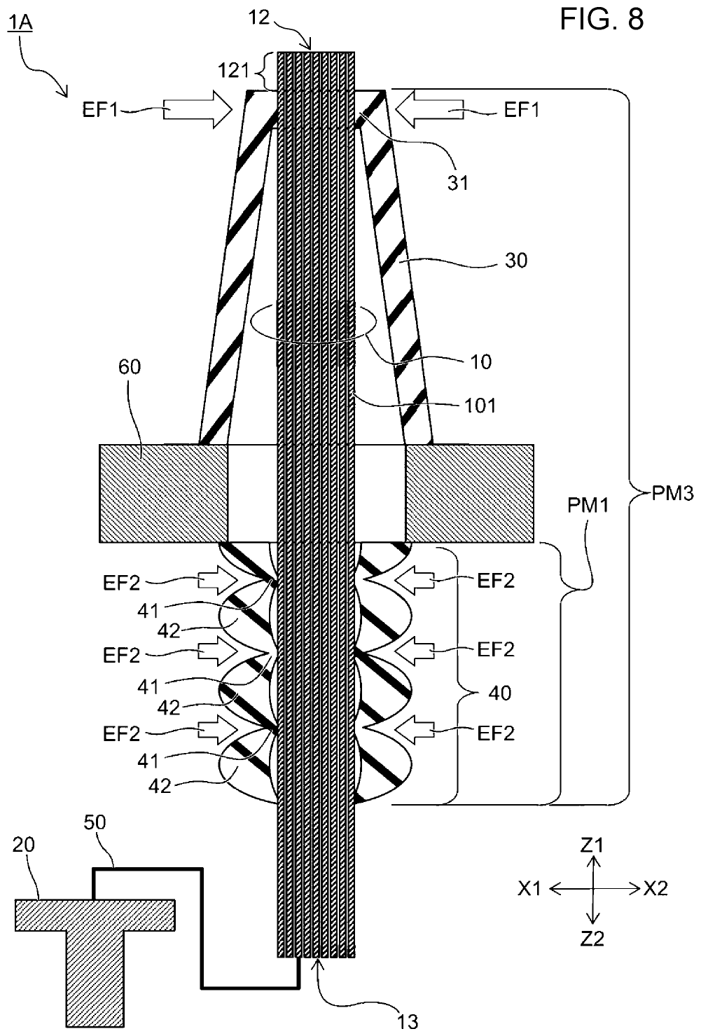

FIG. 8 is a cross-sectional view of a biometric-information measuring electrode 1A according to a second embodiment of the present invention conceptually illustrating the structure. The biometric-information measuring electrode 1A according to the second embodiment of the present invention illustrated in FIG. 8 differs from the biometric-information measuring electrode 1 according to the first embodiment in that the terminal 20 and the second elastic member 40 are not connected and that the rear end 13 of the conductive bundle 10 (the columnar conductor) protrudes from the second elastic member 40 to the Z2 side in the Z1-Z2 direction, but the others are common to the first embodiment. Thus, the second embodiment of the present invention will be described focusing on the difference.

In the biometric-information measuring electrode 1A according to the second embodiment of the present invention, the relative position of the base member 60 and the terminal 20 is fixed by a framework (not illustrated). Thus, in the normal mode, the relative position of the conductive bundle 10 to the terminal 20 is fixed by the support portion 31 supporting the conductive bundle 10.

In the biometric-information measuring electrode 1A, the contact portion 121 of the conductive bundle 10 can be protruded to the Z1 side in the Z1-Z2 direction (toward the distal end 12) by the first method and the third method described in the refresh processing of the biometric-information measuring electrode 1. Accordingly, the biometric-information measuring electrode 1A includes the first protrusion mechanism PM1 and the third protrusion mechanism PM3.

### Third Embodiment

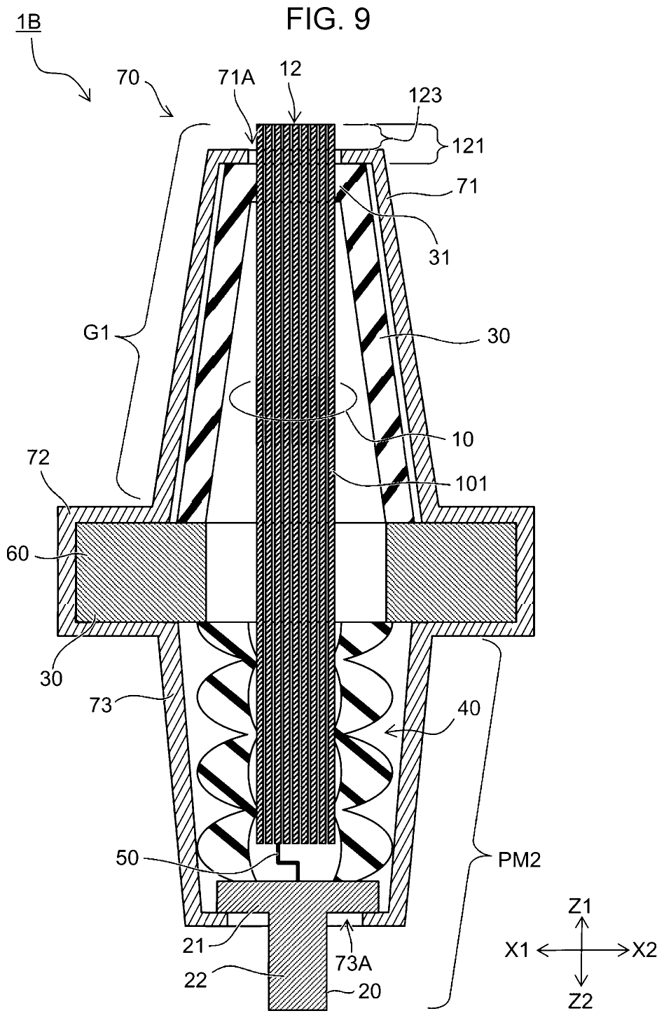

FIG. 9 is a cross-sectional view of a biometric-information measuring electrode 1B according to a third embodiment of the present invention conceptually illustrating the structure. The biometric-information measuring electrode 1B according to the third embodiment of the present invention illustrated in FIG. 9 includes the biometric-information measuring electrode 1 according to the first embodiment and further includes a conductive casing 70 that houses most of the biometric-information measuring electrode 1B. Thus, the third embodiment of the present invention will be described focusing on the difference, or the casing 70.

The casing 70 of the biometric-information measuring electrode 1B may be composed of an electrically conductive material. Examples include a metal-based material, such as copper, and a conductive carbon material. The casing 70 includes an electrode-side casing portion 71, a central casing portion 72, and a terminal-side casing portion 73 which are provided continuously from the Z1 side in the Z1-Z2 direction to the Z2 side in the Z1-Z2 direction.

The electrode-side casing portion 71 of the casing 70 is of substantially truncated cone shape in external form, houses the first elastic member 30 in a hollow passing therethrough in the Z1-Z2 direction, and has an opening 71A, on the Z1 side in the Z1-Z2 direction, connecting to the hollow, as illustrated in FIG. 9. The electrode-side casing portion 71 (the casing 70) is disposed so as to cover the periphery a portion of the conductive bundle 10 (the columnar conductor) adjacent to the distal end 12 in such a manner that the support portion 31 that rotatably supports the conductive bundle 10 is located inside the opening 71A. The electrode-side casing portion 71 has a shape extending along the conductive bundle 10 (in the Z1-Z2 direction). The conductive bundle 10 has the distal end 12 exposed from the opening 71A of the electrode-side casing portion 71 and includes a protruding portion 123 protruding toward the distal end 12 (to the Z1 side in the Z1-Z2 direction). The protruding portion 123 is part of the contact portion 121 of the conductive bundle 10 on the Z1 side in the Z1-Z2 direction (on the distal end 12 side).

The casing 70 (specifically, the electrode-side casing portion 71) covering the periphery of the contact portion 121 of the conductive bundle 10 prevents a portion of the conductive bundle 10 other than the contact portion 121 for use in measurement from coming into contact with the object to be measured including skin (living organism). Thus, this configuration allows repeated use of the biometric-information measuring electrode 1B favorably from a hygiene viewpoint.

In the biometric-information measuring electrode 1B, the electrode-side casing portion 71 and the protruding portion 123 protruding from the electrode-side casing portion 71 to the Z1 side in the Z1-Z2 direction constitute an electrode pin G1. The structure of the electrode pin G1 makes it easy to bring the biometric-information measuring electrode 1B into contact with the target living organism (skin). Thus, the biometric-information measuring electrode 1B with this configuration is easy to increase in the measurement stability.

The electrode-side casing portion 71 may have a tapered shape whose outer shape decreases toward the distal end 12 (to the Z1 side in the Z1-Z2 direction). The tapered shape makes it easy to bring the distal end 12 (the Z1 side in the Z1-Z2 direction) into contact with the target living organism (skin). Thus, the biometric-information measuring electrode 1B with this configuration is further easier to increase in the measurement stability.

The central casing portion 72 of the casing 70 is disc-shaped and having a hollow passing therethrough in the Z1-Z2 direction, in which the base member 60 is housed as illustrated in FIG. 9.

The terminal-side casing portion 73 of the casing 70 is of substantially truncated cone shape in external form, houses the second elastic member 40 in a hollow passing therethrough in the Z1-Z2 direction, and has an opening 73A, on the Z2 side in the Z1-Z2 direction, as illustrated in FIG. 9. The main body 22 of the terminal 20 protrudes through opening 73A. The flange 21 of the terminal 20 is urged to the Z2 side in the Z1-Z2 direction owing to the elastic recovery force of the second elastic member 40 into contact with the periphery of the opening 73A on the inner wall of the terminal-side casing portion 73. Thus, the position of the terminal 20 in the normal mode is fixed by the second elastic member 40 and the terminal-side casing portion 73. In the refresh processing, by pressing the main body 22 of the terminal 20 to the Z1 side in the Z1-Z2 direction, the second method is performed so that the contact portion 121 of the conductive bundle 10 can be protruded to the Z1 side in the Z1-Z2 direction. Thus, the biometric-information measuring electrode 1B includes the second protrusion mechanism PM2, and the second protrusion mechanism PM2 includes the entire terminal 20.

Since the casing 70 has electrical conductivity and is also electrically connected to the terminal 20, signals from the conductive bundle 10 can be transmitted to the terminal 20 with more stability. Thus, the biometric-information measuring electrode 1B with this configuration is easy to increase in the measurement stability. In particular, when the first elastic member 30 has electrical conductivity, the electrical connection between the conductive bundle 10 and the electrode-side casing portion 71 is stably established.

Since the biometric-information measuring electrode 1B includes the casing 70 covering most of the biometric-information measuring electrode 1B, the components of the biometric-information measuring electrode 1B, specifically, the first elastic member 30, the base member 60, the second elastic member 40, and the terminal 20, may not be non-detachably connected. If the first elastic member 30 and the terminal 20 are urged to the inner wall of the casing 70 in the Z1-Z2 direction owing to the elastic recovery force of the first elastic member 30 and the second elastic member 40 in the Z1-Z2 direction, the relative position of the conductive bundle 10 and the terminal 20 can be kept.

Although the casing 70 of the biometric-information measuring electrode 1B includes the electrode-side casing portion 71, the central casing portion 72, and the terminal-side casing portion 73, this is not intended to limit the present invention. The casing 70 may include only the electrode-side casing portion 71 without the central casing portion 72.

### Fourth Embodiment

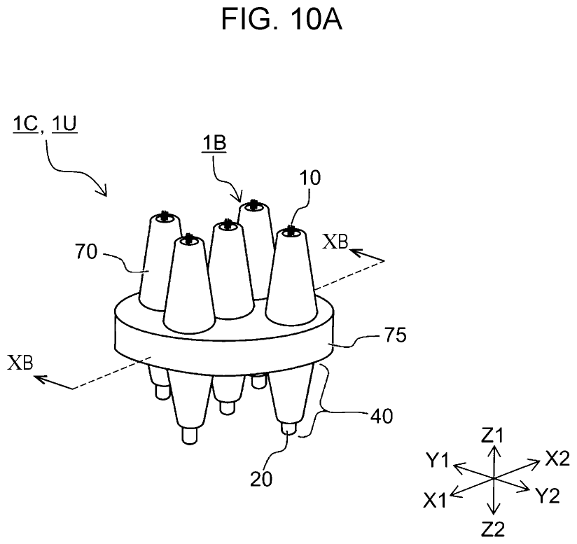

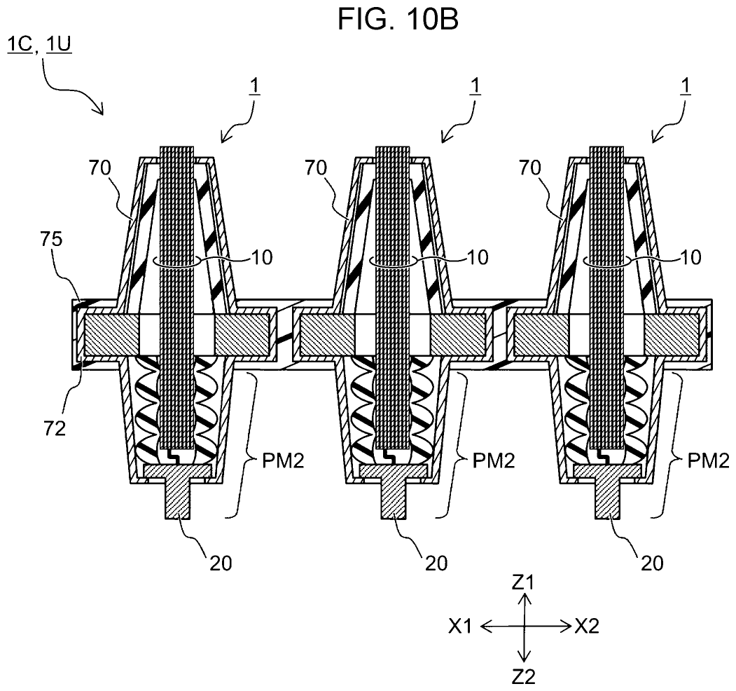

FIG. 10A is an external view of a biometric-information measuring electrode 10 according to a fourth embodiment of the present invention conceptually illustrating the structure. FIG. 10B is a cross-sectional view taken along line XB-XB of FIG. 10A. Since the biometric-information measuring electrode 10 according to the fourth embodiment of the present invention includes a plurality of the biometric-information measuring electrodes 1B according to the third embodiment, the biometric-information measuring electrode 10 is referred to as “electrode unit 1U”.

As illustrated in FIGS. 10A and 10B, the electrode unit 1U (the biometric-information measuring electrode 10) according to the fourth embodiment of the present invention includes five biometric-information measuring electrodes 1B disposed in a common X-Y plane. These biometric-information measuring electrodes 1B are integrated using a resin member 75 covering the central casing portions 72 of the casings 70. The relative position of the plurality of biometric-information measuring electrodes 1B is fixed by the resin member 75. Accordingly, the conductive bundle 10 (the columnar conductor), supported by the support portion 31, of each of the biometric-information measuring electrodes 1B of the electrode unit 1U is electrically connected to the terminal 20.

In the fourth embodiment of the present invention, the protrusion mechanism (the second protrusion mechanism PM2) is provided in each of the plurality of casings 70, so that each second protrusion mechanism PM2 functions independently. This allows the biometric-information measuring electrodes 1B of the electrode unit 1U to function independently. With such a configuration, the plurality of biometric-information measuring electrodes 1B having the same function are independent from each other. Therefore, even if any of the plurality of biometric-information measuring electrodes 1B of the electrode unit 1U fails in operation, the measurement of biometric information can be continued by the remaining biometric-information measuring electrodes 1B. Thus, the electrode unit 1U has high measurement stability.

### Fifth Embodiment

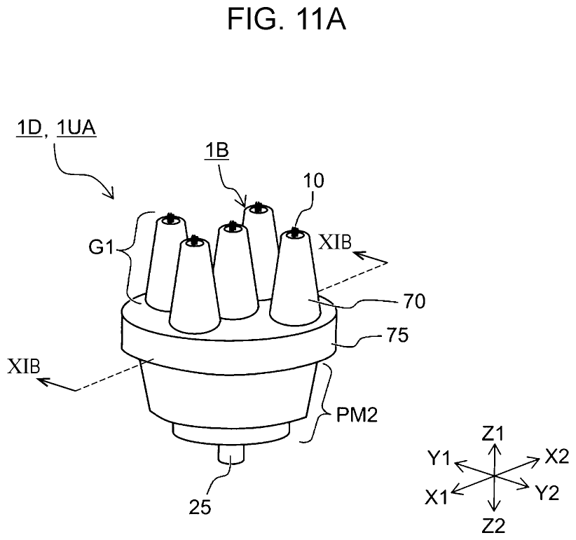

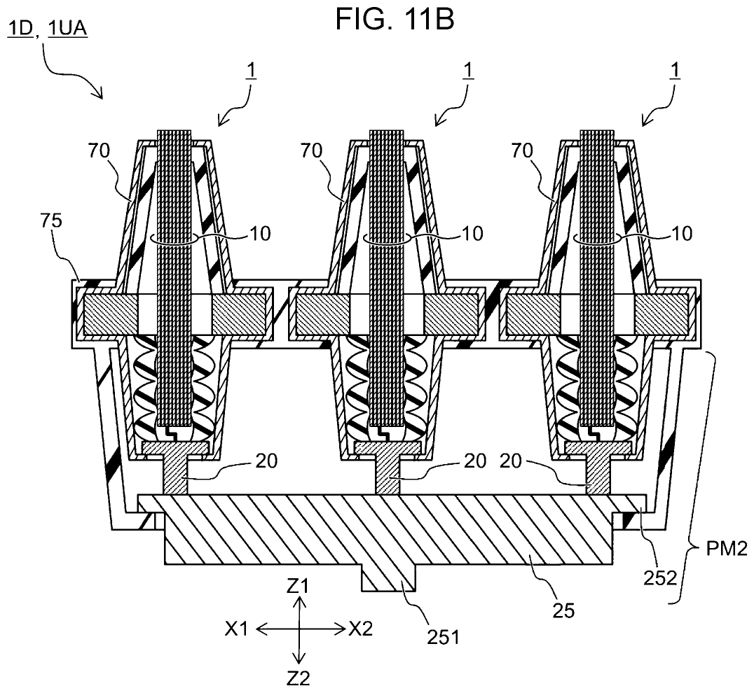

FIG. 11A is an external view of a biometric-information measuring electrode 1D according to a fifth embodiment of the present invention conceptually illustrating the structure. FIG. 11B is a cross-sectional view taken along line XIB-XIB of FIG. 11A. Since the biometric-information measuring electrode 1D according to the fifth embodiment of the present invention includes a plurality of the biometric-information measuring electrodes 1B according to the third embodiment, the biometric-information measuring electrode 1D is referred to as “electrode unit 1UA”, like the electrode unit 1U of the fourth embodiment. The electrode unit 1UA (the biometric-information measuring electrode 1D) according to the fifth embodiment of the present invention has five biometric-information measuring electrodes 1B fixed by the resin member 75, like the electrode unit 1U according to the fourth embodiment. The electrode unit 1UA differs from the electrode unit 1U in that the resin member 75 houses not only the central casing portion 72 but also the terminal-side casing portion 73 in its hollow and that an integrated terminal 25 electrically connected to the terminal 20 of each of the plurality of biometric-information measuring electrodes 1B is provided.

As illustrated in FIG. 11A, the electrode unit 1UA includes five electrode pins G1 protruding from the resin member 75 to the Z1 side in the Z1-Z2 direction, and the integrated terminal 25 protrudes to the Z2 side in the Z1-Z2 direction. As illustrated in FIG. 11B, the integrated terminal 25 includes a protruding portion 251 on the Z2 side in the Z1-Z2 direction for transmitting electrical signals to an external device and a flange 252, on the Z1 side in the Z1-Z2 direction, protruding in the X-Y plane. The terminal 20 of each biometric-information measuring electrode 1B is in contact with an end face of the flange 252 on the Z1 side in the Z1-Z2 direction to establish electrical connection between the terminal 20 and the integrated terminal 25. The surface of the periphery of the flange 252 on the Z2 side in the Z1-Z2 direction is in contact with the inner wall forming the hollow of the resin member 75. In the thus-configured electrode unit 1UA, the elastic recovery force of the second elastic member 40 of the biometric-information measuring electrode 1B (see FIG. 9) to the Z2 side in the Z1-Z2 direction causes the contact pressure between the terminal 20 and the flange 252 and the contact pressure between the flange 252 and the resin member 75.

Accordingly, in order to protrude the conductive bundle 10 (the columnar conductor) of each of the plurality of biometric-information measuring electrodes 1B of the electrode unit 1UA, the integrated terminal 25 is pressed to the Z1 side in the Z1-Z2 direction, so that the terminal 20 of each of the plurality of biometric-information measuring electrodes 1B of the electrode unit 1UA is cooperatively moved to the Z1 side in the Z1-Z2 direction. In other words, the protrusion mechanism of the electrode unit 1UA is the second protrusion mechanism PM2, which is the protrusion mechanism of the biometric-information measuring electrode 1B, and the components of the second protrusion mechanism PM2 include the integrated terminal 25. This configuration allows at least two of the plurality of conductive bundles 10 of the electrode unit 1UA to be cooperatively protruded at the same time. Thus, this configuration reduces the preparation time for the measurement, specifically, operation time for the refresh processing, thus increasing the measurement efficiency.

### Sixth Embodiment

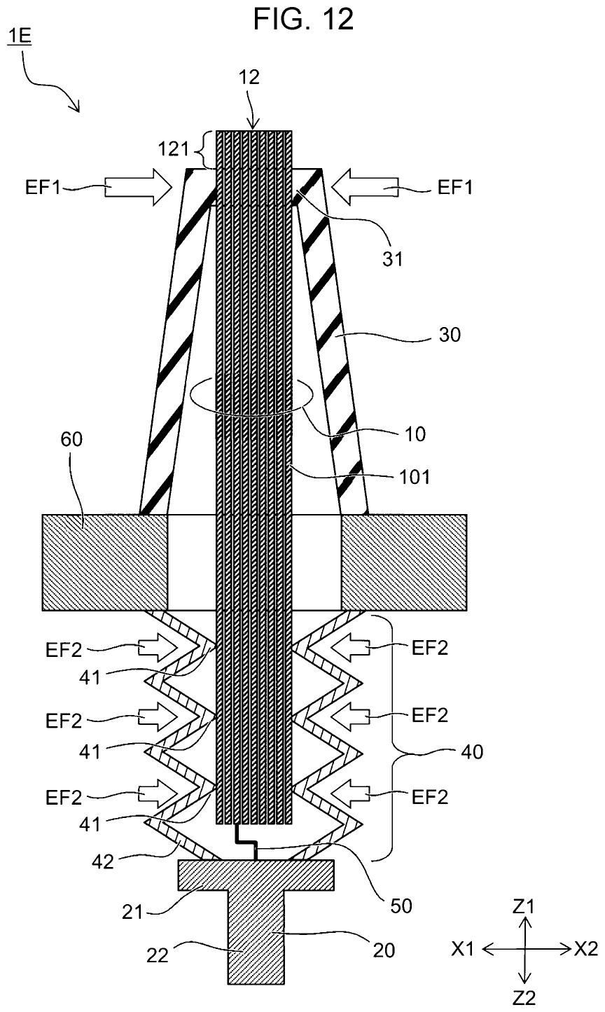

FIG. 12 is a cross-sectional view of a biometric-information measuring electrode 1E according to a sixth embodiment of the present invention conceptually illustrating the structure. As illustrated in FIG. 12, the basic structure of the biometric-information measuring electrode 1E according to the sixth embodiment of the present invention is common to that of the biometric-information measuring electrode 1 according to the first embodiment, but the second elastic member 40 may have a bellows structure obtained by folding metal. Portions of the inner wall of the bellows structure protruding to the center constitute the temporarily holding portions 41, and the main body of the bellows structure constitutes the displacement portions 42. The biometric-information measuring electrode 1E is capable of refresh processing as the biometric-information measuring electrode 1 is, in which any of the first method, the second method, and the third method can be performed.

### Seventh Embodiment

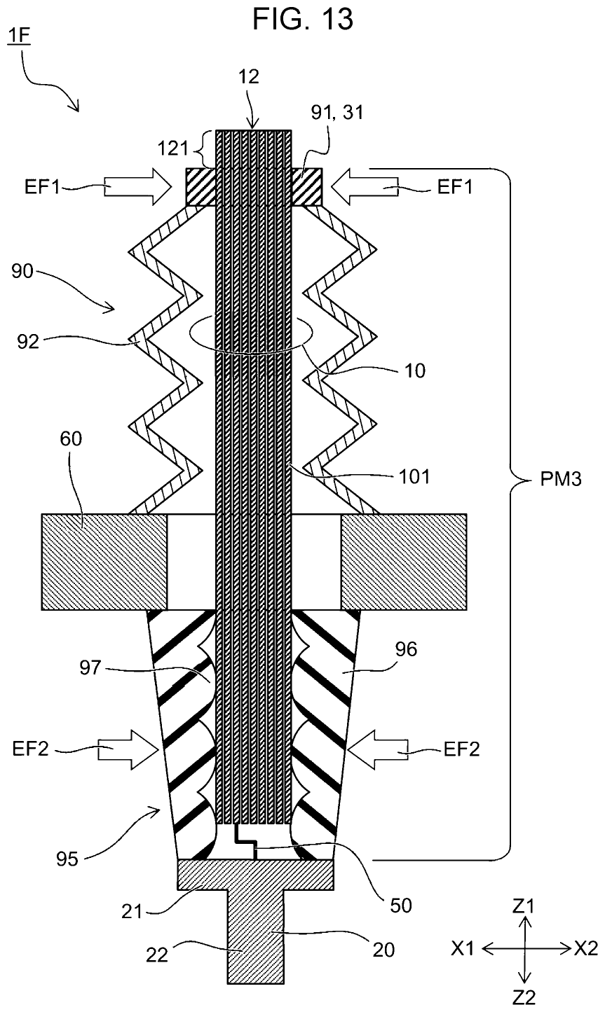

FIG. 13 is a cross-sectional view of a biometric-information measuring electrode 1F according to a seventh embodiment of the present invention conceptually illustrating the structure. As illustrated in FIG. 13, the biometric-information measuring electrode 1F according to the seventh embodiment of the present invention includes a third elastic member 90 instead of the first elastic member 30 of the biometric-information measuring electrode 1. The third elastic member 90 includes an elastic portion 91 composed of an elastic material such as rubber and constituting the support portion 31 that supports the conductive bundle 10 (the columnar conductor) using the first supporting force EF1 and a stretch portion 92 having a bellows structure obtained by folding metal. The stretch portion 92 can stretch along the conductive bundle 10, in other words, can be reversibly elastically displaced in the Z1-Z2 direction.

The biometric-information measuring electrode 1F further includes a fourth elastic member 95 instead of the second elastic member 40 of the biometric-information measuring electrode 1. The fourth elastic member 95 has a tubular shape having a hollow extending in the Z1-Z2 direction and a plurality of protrusions protruding toward the central axis from the inner wall of a main body 96 composed of an elastic material. These protrusions constitute temporarily holding portions 97. Also, the fourth elastic member 95 supports the conductive bundle 10 at the temporarily holding portions 97 using the second supporting forces EF2 lower than the first supporting force EF1. The flange 21 of the terminal 20 connects to an end face of the fourth elastic member 95 on the Z2 side in the Z1-Z2 direction.

The biometric-information measuring electrode 1F is capable of refresh processing as the biometric-information measuring electrode 1 is, in which any of the first method, the second method, and the third method can be performed. A specific example of the refresh processing using the third method will be described. Accordingly, FIG. 13 illustrates the third protrusion mechanism PM3 constituted by the portion from the third elastic member 90 to the fourth elastic member 95.

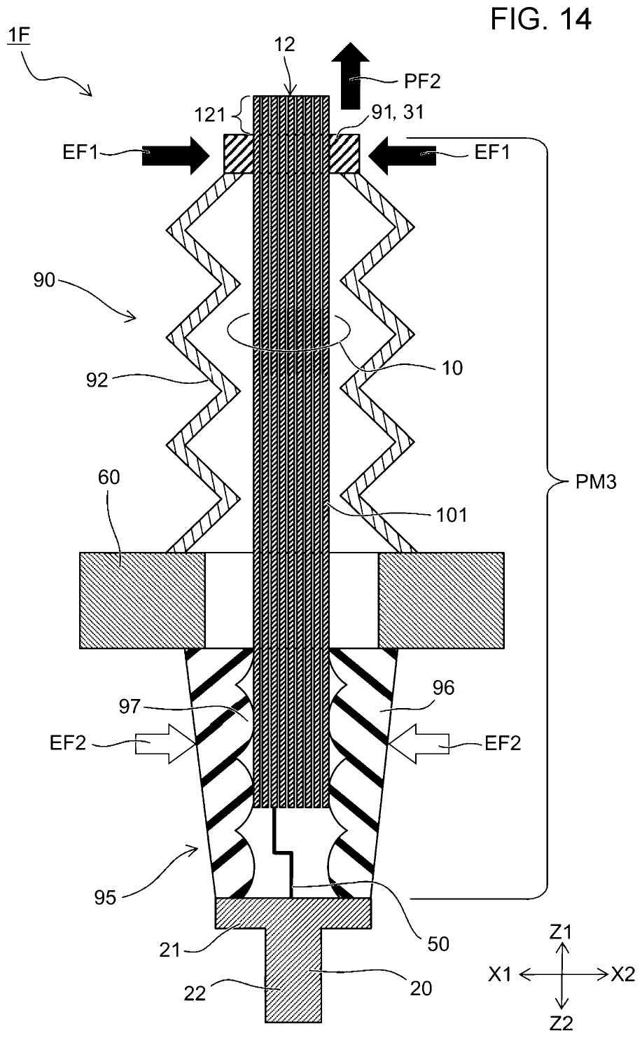

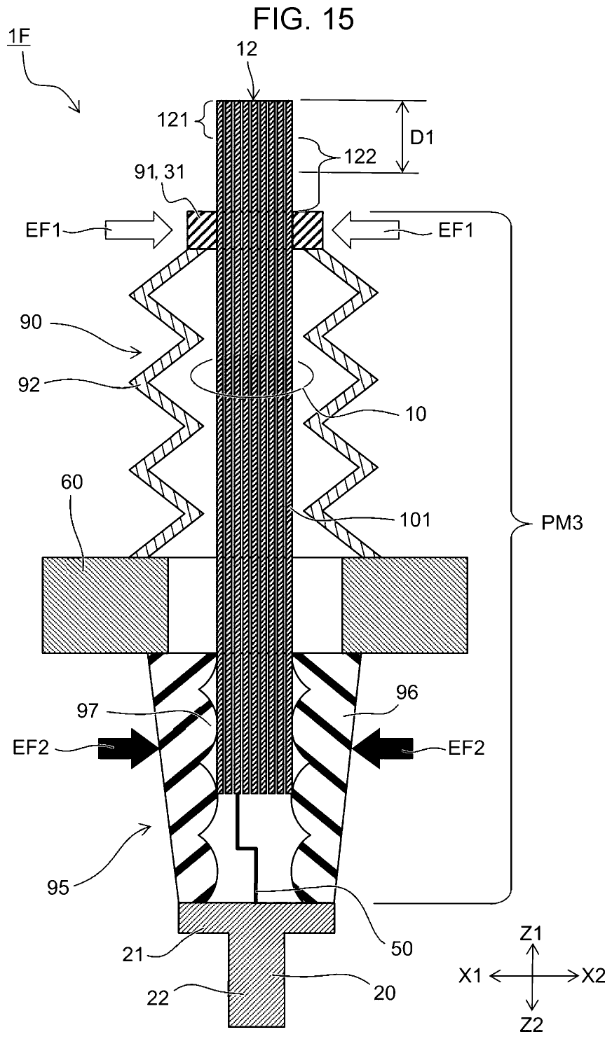

FIG. 14 is a cross-sectional view of the biometric-information measuring electrode 1F according to the seventh embodiment of the present invention conceptually illustrating a state in which the protrusion mechanism (the third protrusion mechanism PM3) is being operated. FIG. 15 is a cross-sectional view of the biometric-information measuring electrode 1F according to the seventh embodiment of the present invention illustrating a state in which the conductive bundle 10 is protruded by operating the protrusion mechanism (the third protrusion mechanism PM3).

The third elastic member 90 of the biometric-information measuring electrode 1F can hold the conductive bundle 10 stronger than the temporarily holding portion 97 of the fourth elastic member 95 with the support portion 31 constituted by the elastic portion 91 when the stretch portion 92 is stretched. In FIG. 14, the arrows representing the first supporting force EF1 of the support portion 31 constituted by the elastic portion 91 are filled in black, which indicates that the first supporting force EF1 becomes stronger than the second supporting forces EF2 at the temporarily holding portions 97 of the fourth elastic member 95. The second external force PF2 directed to the Z1 side in the Z1-Z2 direction is applied to the support portion 31 constituted by the elastic portion 91. As a result, the stretch portion 92 connected to the elastic portion 91 extends in the Z1-Z2 direction. The conductive bundle 10 supported by the first supporting force EF1 also moves to the Z1 side in the Z1-Z2 direction, and at that time, the conductive bundle 10 slides with respect to the temporarily holding portions 97 of the fourth elastic member 95.

Thereafter, as illustrated in FIG. 15, the conductive bundle 10 is held at the temporarily holding portions 97 stronger than at the support portion 31 constituted by the elastic portion 91. In FIG. 15, the arrows representing the second supporting forces EF2 at the temporarily holding portions 97 of the fourth elastic member 95 are filled in black, which indicates that the second supporting forces EF2 become stronger than the first supporting force EF1 at the support portion 31 (the elastic portion 91). In this state, the application of the second external force PF2 is terminated to contract the stretch portion 92 by the elastic recovery of the stretch portion 92. As a result, the conductive bundle 10 slides with respect to the support portion 31 (the elastic portion 91), so that the conductive bundle 10 is moved toward the distal end 12 (to the Z1 side in the Z1-Z2 direction) with respect to the support portion 31 (the elastic portion 91). Thus, the contact portion 121 of the conductive bundle 10 can be protruded from the support portion 31 (the elastic portion 91) toward the distal end 12 (to the Z1 side in the Z1-Z2 direction). By cutting a portion of the conductive bundle 10 including the distal end 12 as in the method illustrated in FIG. 3, a new contact portion 121 of the conductive bundle 10 can be formed, and the refresh processing ends.

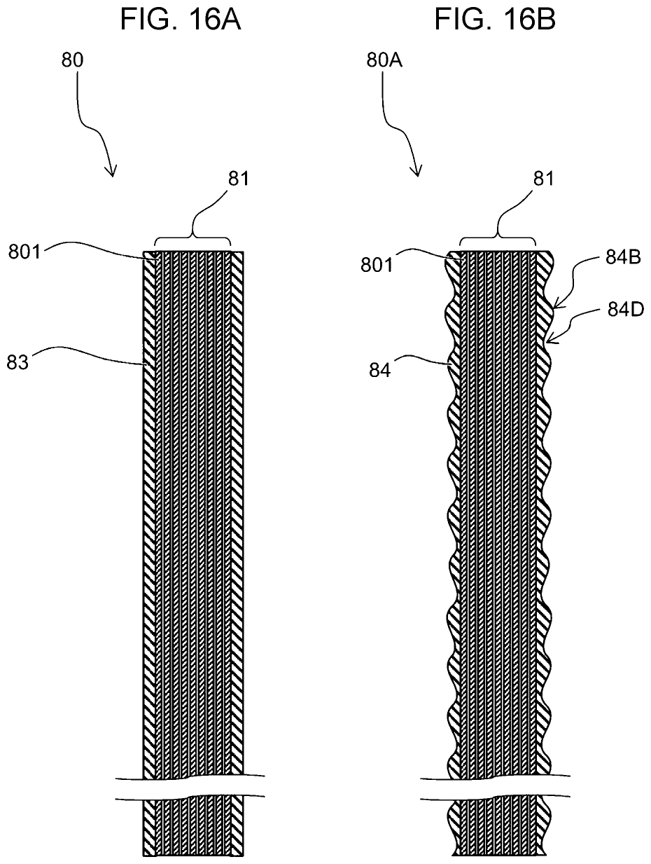

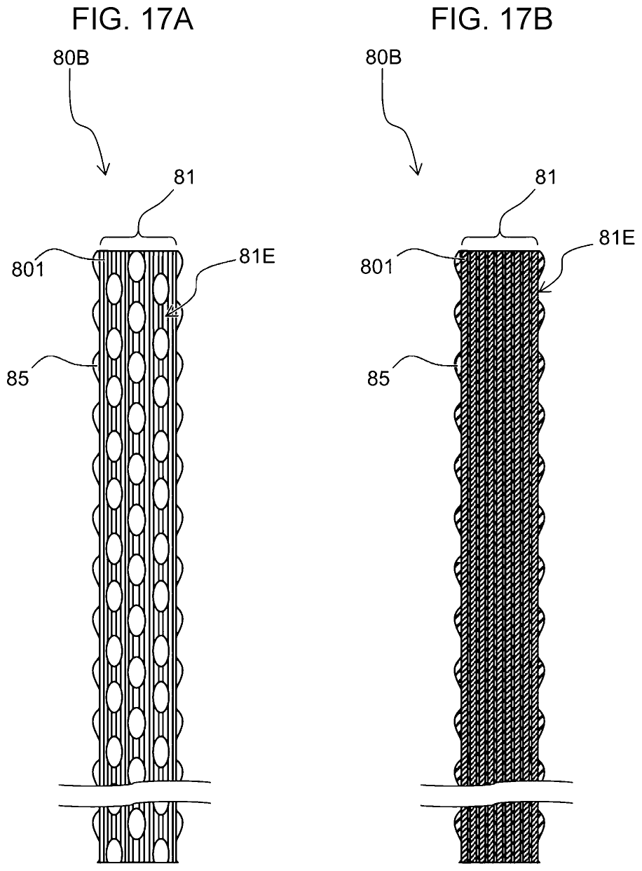

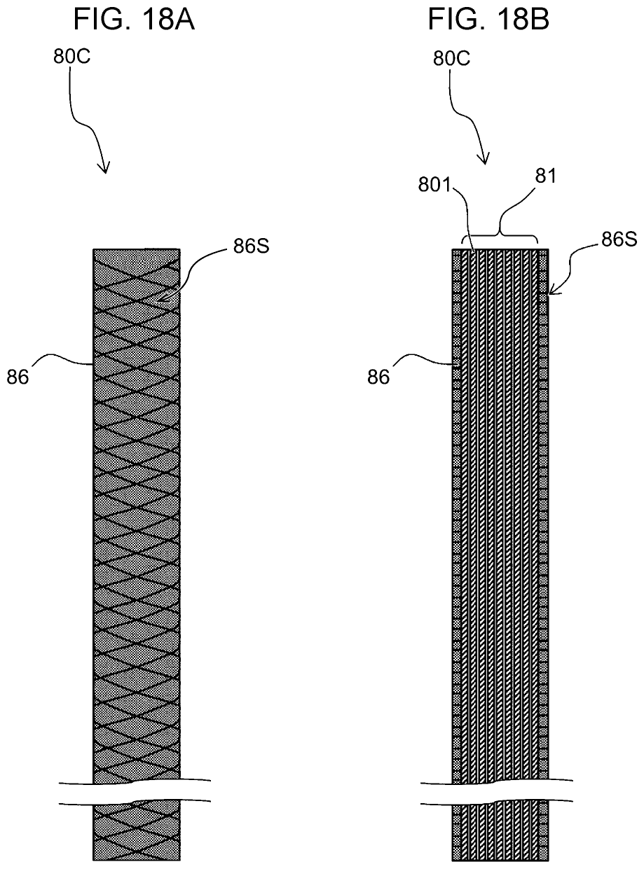

Lastly, a first modification of the conductive bundle 10 that may be included in the biometric-information measuring electrode (1, 1A, 1B, 10, 1D, 1E, or 1F) according to an embodiment of the present invention will be described. FIG. 16A is a cross-sectional view of a coated conductor bundle 80, which is a first modification of the conductive bundle 10, conceptually illustrating an example thereof. FIG. 16B is a cross-sectional view of a coated conductor bundle 80A, which is a second modification of the coated conductor bundle 80, conceptually illustrating the structure thereof. FIG. 17A is an external view of a coated conductor bundle 80B, which is a third modification of the coated conductor bundle 80, conceptually illustrating the structure thereof. FIG. 17B is a conceptual cross-sectional view of the coated conductor bundle 80B (the third modification). FIG. 18A is an external view of a coated conductor bundle 80C, which is a fourth modification of the coated conductor bundle 80, conceptually illustrating the structure thereof. FIG. 18B is a conceptual cross-sectional view of the coated conductor bundle 80C (the fourth modification).

### First Modification

The coated conductor bundle 80 illustrated in FIG. 16A may include a coating material 83 composed of an adhesive material covering a bundle 81 of a plurality of conductive wires 801 so as to bind the conductive wires 801. This coating material 83 allows the bundle form of the plurality of conductive wires 801 to be maintained. The material of the coating material 83 is not limited to particular materials. Since the coating material 83 may be an insulating material, as will be described below, synthetic resin such as polyethylene is given as an example.

The coated conductor bundle 80 with this structure may be used instead of the conductive bundle 10 used in the biometric-information measuring electrodes 1, 1A, 1B, 1E, and 1F and the electrode units 1U (the biometric-information measuring electrode 10) and 1UA (the biometric-information measuring electrode 1D). The coated conductor bundle 80 is easy to maintain its cross-sectional shape and is hardly separated into the plurality of conductive wires 801 in use. Accordingly, the biometric-information measuring electrodes 1, 1A, 1B, 10, and 1D, and the electrode units 1U (the biometric-information measuring electrode 1E) and 1UA (the biometric-information measuring electrode 1F) including the coated conductor bundle 80 has high measurement stability.

In the case of including the coated conductor bundle 80, the coated conductor bundle 80 slides with respect to the support portion 31 supporting the coated conductor bundle 80 with the first supporting force EF1 in the refresh processing, so that the coating material 83 is stripped and stripped off. Thus, a portion of the coated conductor bundle 80 projecting to the Z1 side in the Z1-Z2 direction, that is, the contact portion 121 includes only the bundle 81 of the plurality of conductive wires 801 after sliding on the support portion 31. Therefore, even if the coating material 83 is composed of an insulating material such as synthetic resin, the portion of the coated conductor bundle 80 located at the contact portion 121 has electrical conductivity.

### Second Modification

The coated conductor bundle 80A illustrated in FIG. 16B includes a coating material 84 having uneven thickness areas. The coating material 84 is formed so as to cover the entire outer surface of the bundle 81 of the plurality of conductive wires 801, like the coating material 83 of the coated conductor bundle 80 illustrated in FIG. 16A, and has thick wall portions 84B and thin wall portions 84D alternately disposed in the direction of the long axis of the coated conductor bundle 80A (the Z1-Z2 direction). The difference in the thickness of the coating material 84 makes it easy to strip from the outer surface of the bundle 81 of the plurality of conductive wires 801 while the coated conductor bundle 80A slides with respect to the support portion 31 in the refresh processing.

### Third Modification

A coating material 85 of the coated conductor bundle 80B illustrated in FIGS. 17A and 17B has uneven thickness areas including portions in which the material of the coating material 85 is not provided on the outer surface of the bundle 81 of the plurality of conductive wires 801, that is, portions 81E from which the outer surface of the bundle 81 of the conductive wires 801 is exposed. With this configuration, the members constituting the coating material 85 are disposed in islands on the outer surface of the bundle 81. This allows the coating material 85 to be easily stripped from the outer surface of the bundle 81 of the plurality of conductive wires 801 when the coated conductor bundle 80B slides with respect to the support portion 31.

### Fourth Modification

A coating material 86 of the coated conductor bundle 80C illustrated in FIGS. 18A and 18B includes slit portions 86S that are slit in the thickness direction. With this configuration, the members constituting the coating material 86 are disposed close to each other but independently from each other on the outer surface of the bundle 81. This makes it easy for the coating material 86 to be stripped from the outer surface of the bundle 81 of the plurality of conductive wires 801 when the coated conductor bundle 80C slides with respect to the support portion 31 in the refresh processing.

### Eighth Embodiment

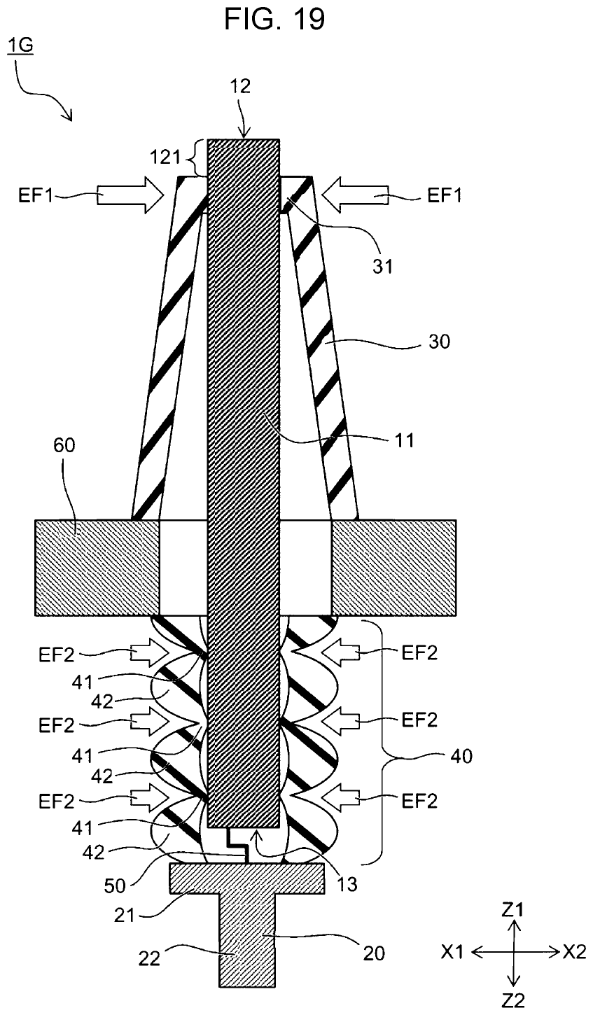

FIG. 19 is a cross-sectional view of a biometric-information measuring electrode 1G according to an eighth embodiment of the present invention conceptually illustrating the structure thereof. The biometric-information measuring electrode 1G illustrated in FIG. 19 differs from the biometric-information measuring electrode 1 according to the first embodiment in including a conductive resin member 11 as a columnar conductor instead of the conductive bundle 10, but the others are common. The biometric-information measuring electrode 1G can perform refresh processing like the biometric-information measuring electrode 1, in which any of the first method, the second method, and the third method can be performed (see FIGS. 1 to 7 and FIGS. 13 to 15). The biometric-information measuring electrodes 1A to 1F (see FIGS. 8 to 13) may include the conductive resin member 11 instead of the conductive bundle 10.

The shape of the conductive resin member 11 is defined by a base material (matrix resin). The base material is synthetic resin. The synthetic resin contains a conductive carbon material and a binder resin that binds the conductive carbon material. The conductive carbon material provides electrical conductivity. In the biometric-information measuring electrode 1, the contact portion 121 at the distal end 12 is conducting to the rear end 13 through the entire conductive resin member 11. This allows biometric information to be collected as electrical signals by bringing the contact portion 121 into contact with the living organism.

A preferable example of the binder resin for binding the carbon material is a thermoplastic resin. Examples of the thermoplastic resin includes, but is not limited to, polyamide (for example, nylon-6 and nylon-66), polycarbonate, polyoxymethylene, polyphenylene sulfide, polyphenylene ether, polyester (for example, polyethylene terephthalate, polybutylene terephthalate, and polyethylene naphthalate), polyethylene, polypropylene, polystyrene, polymethylmethacrylate, an acrylonitrile-styrene (AS) resin, and an acrylonitrile-butadiene-styrene (ABS) resin. They may be used alone or in combination of two or more. Preferable examples of the thermoplastic resin include polyamide, polycarbonate, polyphenylene sulfide, polyester, and polypropylene in the viewpoint of weather resistance, moldability, strength, and cost. Although a preferably binder resin is a thermoplastic resin, a thermosetting resin such as a silicone resin and an urethane resin may be used.

The conductive resin member 11 may be elastically deformable. The elastically deformable conductive resin member 11 can be deformed to correspond to the ununiform shape of the scalp and forehead to come into firm-contact with the scalp and forehead and to suppress the pressure to the scalp and forehead.

The conductive resin member 11 may be provided with a conductive coating at the distal end 12. The conductive coating is disposed to cover at least the distal end of the conductive resin member 11 but is preferably disposed so as to cover the entire conductive resin member 11.

The use of the conductive resin member 11 composed of integrally formed synthetic resin may suppress or reduce generation of pain or a contact mark of the patient, or concentration of force to part of the conductive resin member 11 to damage the conductive resin member 11.

A modification of the conductive resin member 11 of the biometric-information measuring electrode 1G described as the eighth embodiment will be described hereinbelow.

### Fifth Modification

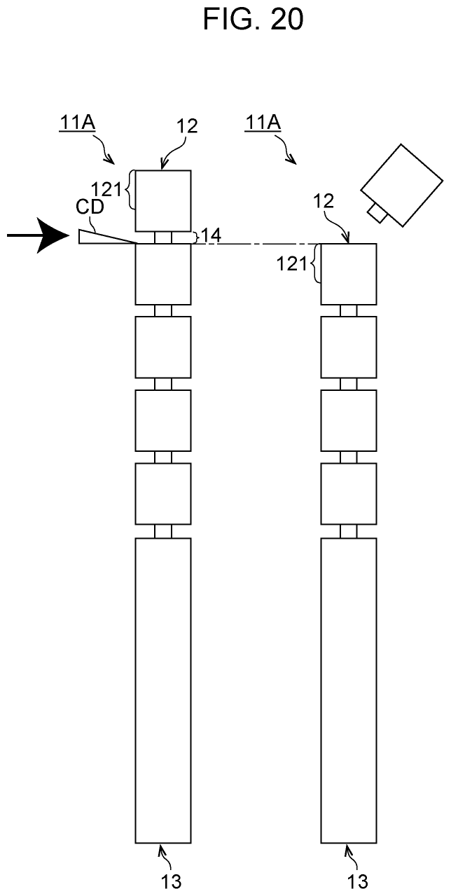

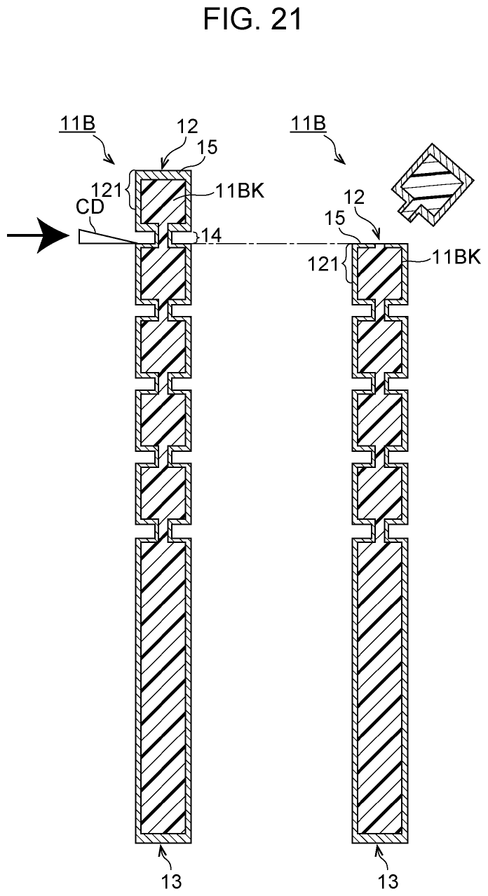

FIG. 20 is a front view of a conductive resin member 11A which is a modification of the conductive resin member 11. FIG. 20 schematically illustrates the conductive resin member 11A before and after refresh processing, in which the diagram on the left illustrates a state before refresh processing, and the diagram on the right illustrates a state after refresh processing.

The conductive resin member 11A is a columnar molding (a columnar conductive resin member) whose base material is synthetic resin and is composed of a conductive synthetic resin containing a conductive carbon material and a binder resin that binds the conductive carbon material, like the conductive resin member 11. The contact portion 121 including the distal end 12 is conducting to the rear end 13 through the conductive resin member 11A.

Since the conductive resin member 11A has notches 14 on the outer peripheral surface, the conductive resin member 11A can be cut to a predetermined length. By cutting the notch 14 at the distal end 12 of the conductive resin member 11A, part of the surface of the newly formed distal end 12 is constituted by part of the surface of the cut notch 14.

### Sixth Modification

FIG. 21 is a cross-sectional view of a conductive resin member 11B, which is a modification of the conductive resin member 11, schematically illustrating the structure thereof. FIG. 21 illustrates the structure of a cross section taken along a plane including the central axis of the conductive resin member 11B. The conductive resin member 11B may have a coated structure including a columnar substrate 11BK and a conductive coating 15 coating the columnar substrate 11BK.

In the conductive resin member 11A of the fifth modification described above, the distal end 12 and the rear end 13 are made conducting by the conductive synthetic resin forming the entire conductive resin member 11A (see FIG. 20). In contrast, in the conductive resin member 11B of this modification, the distal end 12 and the rear end 13 are made conducting by the conductive coating 15 coating the columnar substrate 11BK. The conductive coating 15 preferably coats at least part of the distal end 12 from the viewpoint of reducing the contact impedance of the conductive resin member 11B and the living organism to improve the sensitivity to collect biometric information.

Since the conductive resin member 11B is made conducting by the conductive coating 15, the columnar substrate 11BK can be composed of a nonconductive resin material other than a thermoplastic resin. In this case, the columnar substrate 11BK can be formed using general insulating resin molding, which makes shaping easy. A material, such as an acrylic resin, polyolefin, or polyester, which is insulative but can be used, as a constituent material of the columnar substrate 11BKt, in a manufacturing method capable of shaping, such as injection molding, easily and highly accurately.

The conductive resin member 11B has the conductive coating 15 also on the surface of each notch 14. Therefore, by cutting the notch 14 in the refresh processing, a new surface of the distal end 12 can be formed of the cut surface and part of the surface of the notch 14 coated with the conductive coating 15. Accordingly, at least part of the newly formed distal end 12 is coated with the conductive coating 15. To make the distal end 12 smooth without protrusion, an end of the notch 14 opposite to the distal end 12 to be removed is preferably cut.

As described above, in the conductive resin member 11B, the distal end 12 at least part of which is coated with the conductive coating 15 is formed in the refresh processing. Since the conductive coating 15 is formed on the surface of the distal end 12 in addition to the periphery of the distal end 12, the contact impedance between the contact portion 121 and the living organism can be reduced.

The conductive resin member 11B achieves high electrical conductivity with the configuration in which the surface of the columnar substrate 11BK is coated with the conductive coating 15 while reducing the amount of conductive polymer used. This is economical because the amount of conductive polymer necessary for achieving high electrical conductivity can be smaller than that of a case in which conductive polymer is mixed in the entire conductive resin member.

The conductive coating 15 may contain conductive polymer. Examples of the conductive polymer include PEDOT/PSS in which poly-3,4-ethylenedioxythiophene (PEDOT) is doped with polystyrene sulfonic acid (poly-4-styrene sulfonate [PSS]), polyacetylene, polyaniline, polythiophene, polyphenylene vinylene, and polypyrrole. Among them, PEDOT/PSS is preferably used from the viewpoint of lower contact impedance to living organisms and higher conductivity.

The average thickness of the conductive coating 15 is preferably 1 to 5 μm. Within this range, conductivity necessary to stably convey electrical signals transmitted from the surface of the living organism, such as the scalp, can be obtained, and durability and reduction in material cost and manufacturing cost can also be achieved. The average thickness of the conductive coating 15 refers to the mean value of the thickness of the conductive coating 15. For example, the average thickness is the mean value of the thicknesses of any several measurement points (for example, six measurement points) in a cross section of the conductive coating 15. In the present embodiment, the thickness refers to the length of a layer of the conductive coating 15 perpendicular to the contact surface.

The conductive coating 15 can be formed by applying a solution containing conductive polymer to the surface of the columnar substrate 11BK to form a coated layer and drying and curing the coated layer. Examples of a method for applying a solution containing conductive polymer to the surface of the columnar substrate 11BK include an immersion method of immersing the columnar substrate 11BK in a solution containing conductive polymer and a spray method of spraying a solution containing conductive polymer to the surface of the columnar substrate 11BK. The columnar substrate 11BK may be elastically deformable.

For example, even if the notches 14 of the columnar substrate 11BK are narrow, the width of the notches 14 can be increased by applying a tensile force in the direction of the long axis of the columnar substrate 11BK. Accordingly, forming the conductive coating 15 on the columnar substrate 11BK in its extended state makes it easy to form the conductive coating 15 on the notches 14 of the columnar substrate 11BK when the tensile force is cancelled to bring the columnar substrate 11BK to the shape before being extended.

### Seventh Modification

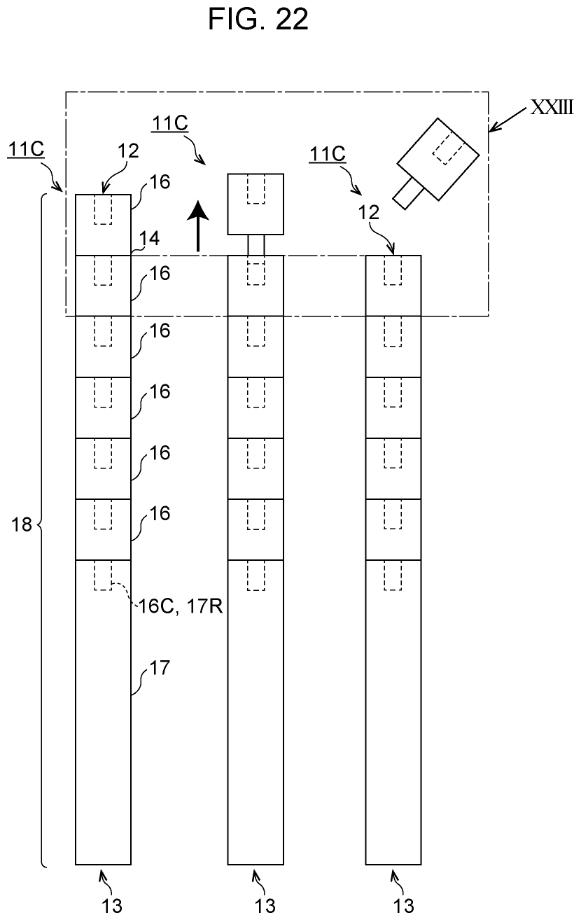

FIG. 22 is a front view of a conductive resin member 11C which is a modification of the conductive resin member 11. FIG. 22 schematically illustrates the conductive resin member 11C in the refresh processing, in which the diagram on the left illustrates a state before the refresh processing, the central diagram illustrates a state during the refresh processing, and the diagram on the right illustrates a state after the refresh processing.

As illustrated in FIG. 22, the conductive resin member 11C may include a combined member 18 in which a plurality of individual contact members 16 and an individual contact member 17 are detachably combined in the direction of the long axis of the conductive resin member C. An individual contact member 16 at the distal end 12 of the conductive resin member 11C may be electrically conducting to the rear end 13 connected to the terminal 20 (see FIG. 19) through the conductive resin member 11C.

By refresh processing for detaching the individual contact member 16 constituting the distal end 12, another individual contact member 16 or 17 can be exposed to form a new distal end 12. In the conductive resin member 11C, the new distal end 12 can be formed without using the cutting device CD (see FIGS. 3 and 21) by separating the individual contact member 16. Thus, since there is no need to cut the conductive resin member 11C, a distal end 12 having no unnecessary protrusion (so-called “burr”) can easily be formed.

The individual contact members 16 may have a fit structure. The individual contact members 16 adjacent in the direction of the long axis of the conductive resin member 11C may be detachably fitted to each other and are connected to the individual contact member 17 having the rear end 13.

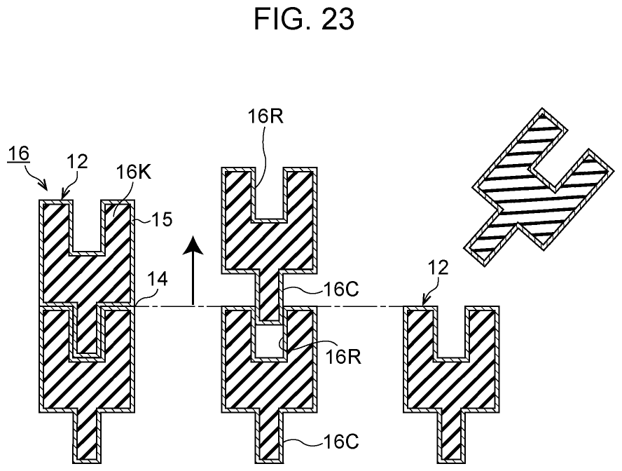

FIG. 23 is a cross-sectional view of a portion enclosed by the one-dot chain line taken along line XXIII in FIG. 22, illustrating, among the plurality of individual contact members 16 constituting the combined member 18, the individual contact member 16 at the distal end 12 and the adjacent individual contact member 16. As illustrated in FIG. 23, each individual contact member 16 has a recess 16R at one end and a protrusion 16C that can be fitted in the recess 16R at the other end. The plurality of individual contact members 16 are detachably combined to each other by the fitting (fit structure) of the recess 16R of the individual contact member 16 and the protrusion 16C of another adjacent individual contact member 16.

As illustrated in FIG. 22, the individual contact member 17 has a recess 17R at an end opposite to the rear end 13. The recess 17R can be fitted on the protrusion 16C of the individual contact member 16. The plurality of individual contact members 16 detachably connected to each other are connected to the electrically conductive individual contact member 17 by the fitting of the protrusion 16C and the recess 17R.

As illustrated in FIG. 23, the entire surface of the base individual substrate 16K of each individual contact member 16 is coated with the conductive coating 15. This allows the distal end 12 of the individual contact member 16 to be conducting to the conductive individual contact member 17 through the contact surface of the conductive coating 15 of the adjacent individual contact member 16.

The notch 14 is a portion of the combined member 18 at which the conductive coatings 15 of the adjacent individual contact members 16, or the conductive coating 15 of the individual contact member 16 and the individual contact member 17 adjacent to each other are in contact. The notch 14 is a contact surface of the conductive coating 15 before the refresh processing, and a portion where a new distal end 12 can be formed after the refresh processing.

Since the individual contact members 16 have the conductive coating 15 described above, the individual substrates 16K need not have electrical conductivity and may be composed of an insulating material. However, the individual substrates 16K are preferably conductive resin members from the viewpoint of increasing the conductivity of the individual contact members 16. The base material of the conductive resin member is a conductive synthetic resin. An example of the conductive resin member is a member containing a conductive carbon material and a binder resin that binds the conductive carbon material.

In the case where the individual substrates 16K of the individual contact members 16 are the conductive resin members, the distal end 12 and the rear end 13 of the individual contact members 16 are made conducting through the combined individual substrates 16K. This eliminates the need for coating the individual substrates 16K with the conductive coating 15. However, it is preferable to dispose the conductive coating 15 so as to coat at least an end of the individual contact member 16 adjacent to the distal end 12 from the viewpoint of decreasing the contact impedance between the distal end 12 of the individual contact member 16 and the living organism to increase the sensitivity of the biometric-information measuring electrode 1G (see FIG. 19).

The combined member 18 may have any other configuration in which the individual contact member 16 at the distal end 12 is detachably combined. Thus, the combined member 18 may not have the configuration illustrated in FIG. 22 in which the plurality of individual contact members 16 with the same shape and one individual contact member 17 that differs in shape from the individual contact members 16 are combined. Other examples other than this configuration include a configuration in which one individual contact member 16 and one individual contact member 17 are combined and a configuration in which a plurality of individual contact members 16 are combined.

### Eighth Modification

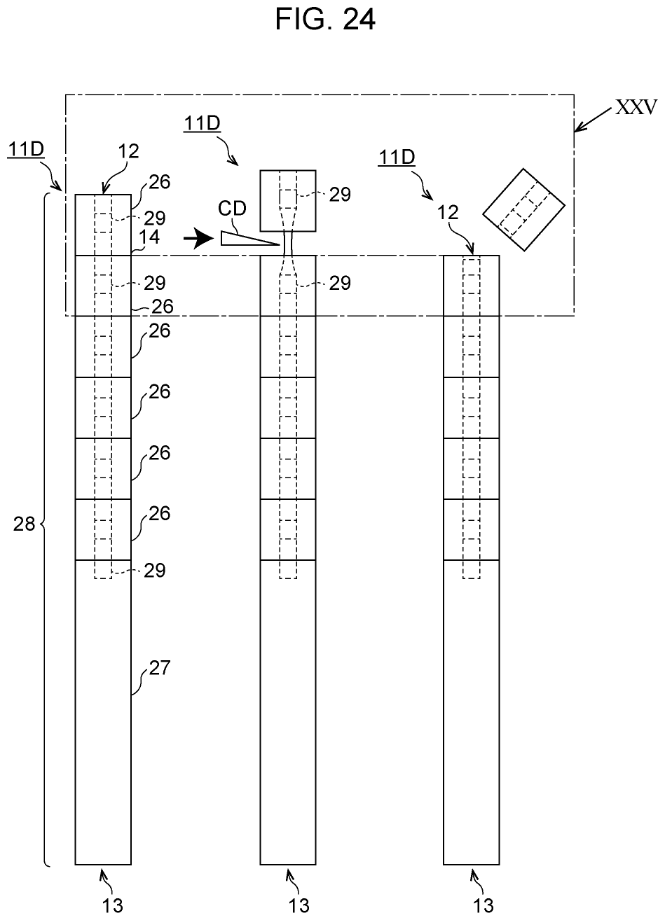

FIG. 24 is a front view of a conductive resin member 11D which is a modification of the conductive resin member 11. FIG. 24 schematically illustrates the conductive resin member 11D in the refresh processing, in which the diagram on the left illustrates a state before the refresh processing, the central diagram illustrates a state during the refresh processing, and the diagram on the right illustrates a state after the refresh processing.

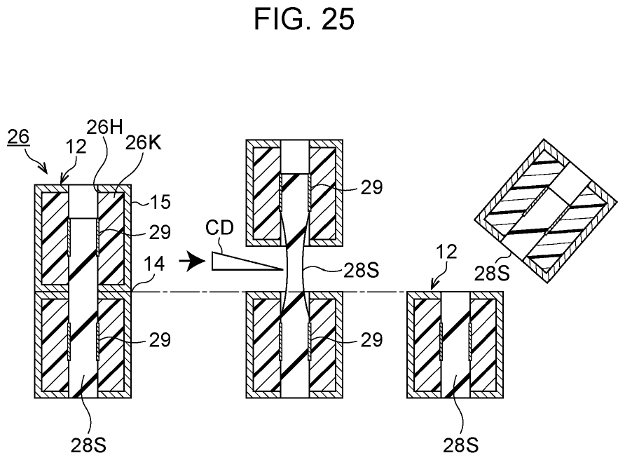

As illustrated in FIG. 24, the conductive resin member 11D includes a combined member 28 in which a plurality of individual contact members 26 and an individual contact member 27 are detachably combined in the direction of the long axis of the conductive resin member 11D. The combined member 28 may include a core material 28S (see FIG. 25). The plurality of individual contact members 26 connected by the core material 28S is attached to the individual contact member 27. The distal end 12 of the conductive resin member 11D is conducting to the rear end 13 connected to the terminal 20 (see FIG. 19) through the conductive resin member 11D.

Each individual contact member 26 is a columnar member having an insertion hole 26H through which the core material 28S extending in the direction of the long axis of the columnar member can be passed. The plurality of individual contact members 26 are connected together, with adjacent individual contact members 26 in contact with each other, by bonding the core material 28S at adhesive portions 29 in the insertion holes 26H of the individual contact members 26, with the core material 28S inserted in the insertion holes 26H.

The core material 28S is an elastic member with the property of extending and contracting in the direction of the long axis of the conductive resin member 11D. This allows the core material 28S between the individual contact member 26 at the distal end 12 of the conductive resin member 11D and the adjacent individual contact member 26 to be cut, with the core material 28S extended in the direction of the long axis of the conductive resin member 11D, in the refresh processing, as illustrated in the central diagram of FIG. 24. Thus, the individual contact member 26 at the distal end 12 side can be removed to form a new distal end 12.

Since the core material 28S is stretchable in the direction of the long axis of the conductive resin member 11D, the core material 28S is contracted after being cut in the refresh processing and is housed in the insertion hole 26H of the individual contact member 26. Thus, the surface of the distal end 12 formed in the refresh processing is smooth without the core material 28S protruded.

FIG. 25 is a cross-sectional view of a portion enclosed by the one-dot chain line XXV in FIG. 24 illustrating, among the plurality of individual contact members 26 constituting the combined member 28, the individual contact member 26 at the distal end 12 and the individual contact member 26 next thereto. As illustrated in FIG. 25, since the surface of the base individual substrate 26K of the individual contact member 26 is coated by the conductive coating 15, the distal end 12 of the individual contact member 26 and the rear end 13 of the individual contact member 27 can be made conducting through the conductive coating 15.

The notch 14 is a portion where the conductive coatings 15 of the adjacent individual contact members 26 are in contact with each other, which is a contact surface of the conductive coating 15 before the refresh processing, and a portion to be a new distal end 12 after the refresh processing.

Since the individual contact members 26 have the conductive coating 15, the individual substrates 26K need not have electrical conductivity. However, the individual substrates 26K are preferably conductive resin members from the viewpoint of increasing the conductivity of the individual contact members 26. In the case where the individual substrates 26K of the individual contact members 26 are the conductive resin members, it is preferable to disposed the conductive coating 15 so as to coat at least an end of the individual contact member 26 adjacent to the distal end 12 from the viewpoint of decreasing the contact impedance between the distal end 12 and the living organism to improve the sensitivity of the biometric-information measuring electrode 1G (see FIG. 19).

While the embodiments have been described, it is to be understood that the present invention is not limited to the embodiments and that addition, deletion, and design change of components, as well as combinations of the features of the embodiments as appropriate, will occur to those skilled in the art without departing from the scope of the present invention.

For example, the biometric-information measuring electrodes 1, 1A, 1B, 10, 1D, 1E (the electrode unit 1U), 1F (the electrode unit 1UA), and 1G include a portion composed of an insulating material, such as the first elastic member 30, but the entire biometric-information measuring electrodes may have conductivity. The elastically deformable material, even with an insulating property, like rubber, can have electrical conductivity by containing an dispersed conductive material, such as a conductive carbon material. Alternatively, an electrically conductive material layer may be formed on the surface of the elastic material. In operation, the temporarily holding portions 41 of the second elastic member 40 may not in contact with the conductive bundle 10, and in performing the first method or the third method, may be brought into contact with the conductive bundle 10.

What is claimed is:

1. A biometric-information measuring electrode comprising:
a columnar conductor having a distal end which is one end and a rear end which is another end;
a terminal electrically connected to the columnar conductor;
a support portion located adjacent to the distal end of the columnar conductor and movably supporting the columnar conductor; and
a protrusion mechanism configured to protrude the columnar conductor toward the distal end,
wherein a portion of the columnar conductor including the distal end is capable of coming into contact with a living organism, and
wherein the support portion keeps a relative position of the columnar conductor to the terminal in a state in which the protrusion mechanism is not performing an operation to protrude the columnar conductor.

2. The biometric-information measuring electrode according to claim 1,
wherein the protrusion mechanism includes:
a temporarily holding portion located nearer to the rear end than the support portion and capable of temporarily holding the columnar conductor; and
a displacement portion capable of changing a separation distance between the support portion and the temporarily holding portion by deforming the displacement portion itself,
wherein the temporarily holding portion holds the columnar conductor stronger than the support portion when the columnar conductor moves toward the distal end in accordance with deformation of the displacement portion.

3. The biometric-information measuring electrode according to claim 2, wherein the displacement portion is movable in an extending direction of the columnar conductor.

4. The biometric-information measuring electrode according to claim 2, wherein the displacement portion includes a portion including an elastic member.

5. The biometric-information measuring electrode according to claim 2, wherein the displacement portion has a bellows structure including a stretch portion extending and contracting along the columnar conductor.

6. The biometric-information measuring electrode according to claim 5, wherein the bellows structure allows the support portion to hold the columnar conductor stronger than the temporarily holding portion when the stretch portion is extended.

7. The biometric-information measuring electrode according to claim 1, further comprising an elastic support portion constituting at least part of the support portion and elastically supporting the columnar conductor.

8. The biometric-information measuring electrode according to claim 1,
wherein the protrusion mechanism includes a pressing member located at a further rear portion than the rear end of the columnar conductor, and
wherein when moving the columnar conductor toward the distal end, the pressing member is capable of coming into contact with the portion of the columnar conductor including the rear end.

9. The biometric-information measuring electrode according to claim 1, further comprising:
at least one casing having an opening through which the portion of the columnar conductor including the distal end is passed and exposed,
wherein the support portion is located inside the opening, and
wherein the casing covers a periphery of the portion of the columnar conductor including the distal end.

10. The biometric-information measuring electrode according to claim 9, wherein the casing has a tapered shape with a smaller outer peripheral shape toward the distal end.

11. The biometric-information measuring electrode according to claim 9,
wherein the casing has a shape extending in a long axis direction of the columnar conductor, and
wherein a protruding portion of the columnar conductor protruding from the casing toward the distal end and the casing constitute an electrode pin.

12. The biometric-information measuring electrode according to claim 9, wherein the casing has electrical conductivity and is electrically connected to the terminal.

13. The biometric-information measuring electrode according to claim 9,
wherein the at least one casing comprises a plurality of casings, and
wherein, in each casing, the columnar conductor supported by the support portion is electrically connected to the terminal.

14. The biometric-information measuring electrode according to claim 13, wherein the protrusion mechanism is provided for each of the plurality of casings.

15. The biometric-information measuring electrode according to claim 13, wherein the protrusion mechanism cooperatively operates at least two of the plurality of columnar conductors corresponding to the plurality of casings.

16. The biometric-information measuring electrode according to claim 1, wherein the columnar conductor includes a portion of a conductive bundle of a plurality of conductive wires.

17. The biometric-information measuring electrode according to claim 16, wherein the conductive bundle includes a portion of the plurality of conductive wires bound with an adhesive material.

18. The biometric-information measuring electrode according to claim 16, wherein the conductive wires include carbon fibers.

19. The biometric-information measuring electrode according to claim 16, wherein at least part of an outer surface of the conductive bundle includes a coating material that binds the plurality of conductive wires.

20. The biometric-information measuring electrode according to claim 19, wherein the coating material adheres to the plurality of conductive wires to such an extent as to be stripped by sliding of the support portion and the conductive bundle performed in the operation of the protrusion mechanism protruding the conductive bundle.

21. The biometric-information measuring electrode according to claim 1,
wherein the columnar conductor comprises a conductive resin member whose base material is synthetic resin, and
wherein a new distal end can be formed by cutting part of the conductive resin member.

22. The biometric-information measuring electrode according to claim 21, wherein the conductive resin member contains a conductive carbon material and a binder resin that binds the conductive carbon material.

23. The biometric-information measuring electrode according to claim 21, wherein the conductive resin member has a conductive coating disposed so as to coat at least an end of the distal end.

24. The biometric-information measuring electrode according to claim 23, wherein the conductive coating is disposed so as to coat the entire conductive resin member.

25. The biometric-information measuring electrode according to claim 1,
wherein the columnar conductor has a notch on its outer peripheral surface, and
wherein, when a portion of the columnar conductor adjacent to the distal end is cut off, part of a surface of the notch constitutes part of a surface of a new distal end formed.

26. The biometric-information measuring electrode according to claim 25,
wherein the columnar conductor includes a columnar conductive resin member whose base material is synthetic resin, and
wherein the notch is provided on the outer peripheral surface of the columnar conductive resin member.

27. The biometric-information measuring electrode according to claim 26, wherein the columnar conductive resin member contains a conductive carbon material and a binder resin that binds the conductive carbon material.

28. The biometric-information measuring electrode according to claim 25,
wherein the columnar conductor has a coated structure including a columnar substrate and a conductive coating that coats the columnar substrate, and
wherein the notch is disposed on the outer peripheral surface of the coated structure.

29. The biometric-information measuring electrode according to claim 1,
wherein the columnar conductor comprises a combined member in which a plurality of individual contact members are detachably combined in a long axis direction of the columnar conductor,
wherein the combined member constitutes a portion of the columnar conductor adjacent to the distal end, and
wherein the individual contact member at the distal end of the combined member is conducting to the terminal.

30. The biometric-information measuring electrode according to claim 29,
wherein the individual contact members of the combined member have a fitted structure, and
wherein the individual contact members adjacent in the long axis direction of the columnar conductor are detachably combined using the fitted structure.

31. The biometric-information measuring electrode according to claim 30,
wherein the combined member includes a core material extending in the long axis direction of the columnar conductor, and
wherein the core material passes through the plurality of individual contact members.

32. The biometric-information measuring electrode according to claim 31, wherein the core material has electrical conductivity.

33. The biometric-information measuring electrode according to claim 29, wherein an individual substrate that is a base of each individual contact member is a conductive resin member whose base material is synthetic resin.

34. The biometric-information measuring electrode according to claim 33, wherein the conductive resin member contains a conductive carbon material and a binder resin that binds the conductive carbon material.

35. The biometric-information measuring electrode according to claim 33, wherein the individual contact member has a conductive coating disposed so as to coat at least an end of the conductive resin member adjacent to the distal end.

36. The biometric-information measuring electrode according to claim 35, wherein the conductive coating is disposed so as to coat the entire conductive resin member.

37. The biometric-information measuring electrode according to claim 29,
wherein an individual substrate that is a base of each individual contact member comprises an insulating material, and
wherein a conductive coating is disposed so as to coat the entire insulating material.

38. The biometric-information measuring electrode according to claim 23, wherein the conductive coating comprises conductive polymer.

39. The biometric-information measuring electrode according to claim 1, wherein the columnar conductor is elastically deformable.

40. A method for measuring biometric information using the biometric-information measuring electrode according to claim 1, the method comprising the steps of:
protruding part of the columnar conductor toward the distal end to form an additional protruding portion by operating the protrusion mechanism;
removing part of the columnar conductor in such a manner that at least part of the additional protruding portion protruded in the protruding step constitutes a cut-remaining component of the biometric-information measuring electrode; and
after the removing step, measuring biometric information by bringing the portion of the columnar conductor protruding toward the distal end into contact with the living organism.
